(19) United States
(12) Patent Application Publication
(10) Pub. No.: US 2025/0160699 A1
(43) Pub. Date: May 22, 2025

(54) AUTOMATED ASSESSMENT OF MICROVASCULAR HEALTH IN SKELETAL MUSCLE

(71) Applicant: The Florida State University Research Foundation, Inc., Tallahassee, FL (US)

(72) Inventors: Judy M. Delp, Tallahassee, FL (US); Xiuwen Liu, Tallahassee, FL (US); Cesar Rodriguez, Tallahassee, FL (US); Cole Smith, Tallahassee, FL (US); Steven Gordon, Tallahassee, FL (US)

(21) Appl. No.: 18/948,415

(22) Filed: Nov. 14, 2024

Related U.S. Application Data
(60) Provisional application No. 63/598,557, filed on Nov. 14, 2023.

(51) Int. Cl. A61B 5/1455; A61B 5/00

(52) U.S. Cl. CPC A61B 5/14551; A61B 5/7264; A61B 5/746; A61B 5/7475

(57) ABSTRACT

An exemplary system and method that non-invasively use re-perfused oxygen saturation signals from near-infrared spectroscopy signal (NIRS) measurement acquired after a microvascular-occluded-induced state to evaluate microvascular health in a subject. The exemplary system and method are configured to use a trained AI model (engineered features or deep learning model), or a classifier derived therefrom, to estimate for a perfusion index (can also be referred to as a reperfusion index due to the occlusion) that can be used to output an indicator for the presence and/or non-presence of microvascular dysfunction in a subject that exhibit abnormal NIRS observation when the subject is subject to an microvascular occlusion induced state. The exemplary system and method can be used to pre-screen for patients with onset microvascular or vascular abnormalities in the limbs to prescribe exercise therapy or drug therapy.

### RELATED APPLICATION

This U.S. application claims priority to, and the benefit of, U.S. Provisional Patent Application No. 63/598,557, filed Nov. 14, 2023, entitled, “AUTOMATED ASSESSMENT OF MICROVASCULAR HEALTH IN SKELETAL MUSCLE,” which is incorporated by reference herein in its entirety.

### BACKGROUND

Accurate evaluation of muscle health is crucial for diagnosing diseases like diabetes and peripheral vascular disease, especially in older adults. Current standards for the evaluation of microvascular health in skeletal muscle include a 6-minute walk test and vascular occlusion test (VOT).

Advanced procedures with magnetic resonance imaging (MRI), assessing tissue oxygenation and perfusion in response to the well-established ischemia-reperfusion paradigm, have revealed significantly impaired lower limb microvascular reactivity in individuals with PAD compared to matched controls with more significant microvascular dysfunction associated with PAD severity. The use of MRI and related imaging techniques to diagnose microvascular dysfunction in the initial assessment of PAD microvascular is nevertheless restricted in many settings due to its limited availability, high time commitment, and cost time.

There would be, therefore, a benefit to improving the assessment of microvascular health in skeletal muscle.

### SUMMARY

An exemplary system and method are disclosed that non-invasively use oxygen saturation signals from near-infrared spectroscopy signal (NIRS) measurement acquired during reperfusion after a microvascular-occluded-induced state to evaluate microvascular health in a subject. Near-infrared spectroscopy (NIRS) employs near-infrared light to measure tissue oxygenation and metabolism in real-time noninvasively. In skeletal muscle, it can assess oxygen delivery and metabolism up to a depth of 2.5 cm. Despite its potential, clinical use of NIRS has been limited by inconsistent protocols and data interpretation. The exemplary system and method are configured to use a trained AI model (engineered features or deep learning model) or a classifier derived therefrom to estimate for a perfusion index (can also be referred to as a reperfusion index due to the occlusion) that can be used to output an indicator for the presence and/or non-presence of microvascular dysfunction in a subject that exhibits abnormal NIRS observation when the subject is subject to a microvascular occlusion induced state. The exemplary system and method can be used to pre-screen patients with onset microvascular or vascular abnormalities in the limbs to prescribe exercise therapy or drug therapy.

The microvascular-occluded-induced state is induced by the application of pressure, e.g., to the thigh region of the subject, e.g., in a vascular occlusion test, to occlude the perfusion of the blood in the capillaries and microvascular structures, e.g., of the lower extremity regions downstream of the occlusion. For the application of pressure to the thigh, e.g., for lower limb assessment, the pressure may be applied at least for 3-5 minutes to cause a substantial reduction in saturation of hemoglobin with oxygen in the extremity. The body's response to the occlusion and the post recovery re-perfusion is measured via near-infrared spectroscopy to provide the indication of microvascular dysfunction or other like or associated dysfunction. In some embodiments, the occlusion and measurement may be performed via vascular occlusion test protocols. Microvascular dysfunction can be used as a proxy for loss of the ability to walk, e.g., as quantified via a 6-minute walk test. The classifier can be configured to output, for a given age patient, the presence of microvascular dysfunction, the non-presence of microvascular dysfunction (e.g., for anomaly detection), a severity score for microvascular dysfunction, an estimated perfusion index or score, an estimate of the likelihood of failing the walking test, an estimated distance to failure in the walking test, or the like.

In some implementations, the exemplary system can also automate the collection of NIRS signals by controlling various external devices to perform a vascular occlusion operation and record NIRS signals.

In some aspects, the techniques described herein relate to a system including: at least one processor; and memory having instructions stored thereon that, when executed by the at least one processor, cause the system to: obtain a re-perfused oxygen saturation signal (e.g., near-infrared spectroscopy signal) recorded from a second position at an extremity of the subject (e.g., during a vascular occlusion operation (e.g., vascular occlusion test) downstream to a first position having positioned a pressure cuff to cause occlusion of the extremity when inflated; generate, via a trained AI model, or a model derived therefrom, a perfusion index value or score, wherein the trained AI model was trained based on a microvascular function assessment and re-perfused oxygen saturation signal; and output the perfusion index value or score or a parameter derived therefrom as an output of the trained AI model, wherein the output is presented in a graphical user interface or report for use by a clinician to diagnose or treat a microvascular dysfunction or disease of the extremity.

In some aspects, the techniques described herein relate to a system, wherein the instructions further cause the system to: cause an auditory or visual alert to be generated at the graphical user interface if a microvascular dysfunction is detected.

In some aspects, the techniques described herein relate to a system, wherein the alert includes an indication of a type of microvascular dysfunction, wherein the type of microvascular dysfunction is predicted from the perfusion index.

In some aspects, the techniques described herein relate to a system, wherein the instructions further cause the system to: preprocess the re-perfused oxygen saturation signal to remove outliers (e.g., using a seven-tap outlier detection filter) prior to using the trained AI model to generate the perfusion index value or score.

In some aspects, the techniques described herein relate to a system, further including: a pressure cuff configured to be positioned at the first position; and direct control output for inflation and deflation of the pressure cuff in accordance with a predefined test procedure (e.g., in a VOT).

In some aspects, the techniques described herein relate to a system, further including: a near-infrared spectroscopy device configured to acquire the re-perfused oxygen saturation signal.

In some aspects, the techniques described herein relate to a system, wherein the trained AI model employs one or more ML features selected from the group consisting of: a first feature associated with a rate of oxygen utilization during a period of occlusion; a second feature associated with a rate of microvascular reperfusion upon release of occlusion; a third feature associated with a hyperemic response of muscle microvasculature; a fourth feature associated with a rate of return of oxygen saturation to baseline after occlusion; and a fifth feature associated with overall oxygenation of muscle during the occlusion of the extremity.

In some aspects, the techniques described herein relate to a system, wherein the trained AI model is a neural network.

In some aspects, the techniques described herein relate to a method for estimating microvascular health in an extremity of a subject, the method including: obtaining by a processor a re-perfused oxygen saturation signal (e.g., near-infrared spectroscopy signal) recorded from a second position at the extremity of the subject (e.g., during a vascular occlusion test (VOT)) downstream to a first position having positioned a pressure cuff to cause occlusion of the extremity when inflated; generating, via a trained AI model, or a model derived therefrom, a perfusion index value or score, wherein the trained AI model was trained based on a microvascular function assessment and re-perfused oxygen saturation signal; and outputting the perfusion index value or score or a parameter derived therefrom as an output of the trained AI model, wherein the output is presented in a graphical user interface or report for use by a clinician to diagnose or treat a microvascular dysfunction or disease of the extremity.

In some aspects, the techniques described herein relate to a method, further including: determining whether the estimated microvascular function is indicative of microvascular dysfunction, wherein the indication includes an alert if microvascular dysfunction is detected.

In some aspects, the techniques described herein relate to a method, wherein the alert includes an indication of a type of microvascular dysfunction, wherein the type of microvascular dysfunction is predicted from the perfusion index.

In some aspects, the techniques described herein relate to a method, further including: preprocessing the re-perfused oxygen saturation signal to remove outliers (e.g., using a seven-tap outlier detection filter) prior to using the trained AI model to generate the perfusion index value or score.

In some aspects, the techniques described herein relate to a method, further including: controlling inflation and deflation of the pressure cuff in accordance with a predefined test procedure.

In some aspects, the techniques described herein relate to a method wherein a near-infrared spectroscopy device acquired the re-perfused oxygen saturation signal.

In some aspects, the techniques described herein relate to a method, wherein the trained AI model employs one or more ML features selected from the group consisting of: a first feature associated with a rate of oxygen utilization during a period of occlusion; a second feature associated with a rate of microvascular reperfusion upon release of occlusion; a third feature associated with a hyperemic response of muscle microvasculature; a fourth feature associated with a rate of return of oxygen saturation to baseline after occlusion; and a fifth feature associated with overall oxygenation of muscle during the occlusion of the extremity.

In some aspects, the techniques described herein relate to a non-transitory computer readable medium having instructions for estimating microvascular health in an extremity of a subject, the instructions when executed by a processor causes the processor to: obtain a re-perfused oxygen saturation signal (e.g., near-infrared spectroscopy signal) recorded from a second position at the extremity of the subject (e.g., during a vascular occlusion test (VOT)) downstream to a first position having positioned a pressure cuff to cause occlusion of the extremity when inflated; generate, via a trained AI model, or a model derived therefrom, a perfusion index value or score, wherein the trained AI model was trained based on a microvascular function assessment and re-perfused oxygen saturation signal; and output the perfusion index value or score or a parameter derived therefrom as an output of the trained AI model, wherein the output is presented in a graphical user interface or report for use by a clinician to diagnose or treat a microvascular dysfunction or disease of the extremity.

In some aspects, the techniques described herein relate to a non-transitory computer-readable medium, wherein the instructions, when executed by the processor, further cause the processor to determine whether the estimated microvascular function is indicative of microvascular dysfunction, wherein the indication includes an alert if microvascular dysfunction is detected.

In some aspects, the techniques described herein relate to a non-transitory computer-readable medium, wherein the alert includes an indication of a type of microvascular dysfunction, wherein the type of microvascular dysfunction is predicted from the perfusion index.

In some aspects, the techniques described herein relate to a non-transitory computer-readable medium, wherein the instructions, when executed by the processor, further cause the processor to control the inflation and deflation of the pressure cuff in accordance with a predefined test procedure.

In some aspects, the techniques described herein relate to a non-transitory computer-readable medium, wherein a near-infrared spectroscopy device acquired the re-perfused oxygen saturation signal.

### BRIEF DESCRIPTION OF THE DRAWINGS

FIG. 1A shows an example system configured to non-invasively use re-perfused oxygen saturation signals from near-infrared spectroscopy signal (NIRS) measurement acquired after a microvascular-occluded-induced state to evaluate microvascular health in a subject in accordance with an illustrative embodiment.

FIG. 1B shows an example re-perfused oxygen saturation signal employed by the system of FIG. 1A, according to some implementations.

FIG. 2 shows an example method of generating the trained AI model, including the signal collection and processing pipeline 200 for evaluating microvascular function in a subject, according to some implementations.

FIG. 3 shows an example NIRS assessment system, according to some implementations.

FIG. 4 shows a process for estimating microvascular function in a subject using NIRS data, according to some implementations.

FIG. 5 shows an example method of analysis of the re-perfused oxygen saturation signal, according to some implementations.

FIGS. 6A-6C are graphs indicating a rate of reperfusion (ΔSmO₂/s) upon release of occlusion during a VOT correlated to Ankle-Brachial Index (ABI) and walking distance measured during a Six-Minute Walk Test (6MWT), according to some implementations.

FIGS. 7A and 7B are graphs indicating an area of hyperemic response post-occlusion correlated with walking distance measured during the 6MWT, according to some implementations.

FIGS. 8A-8C are graphs indicating desaturation rate (ΔStO₂/s) during occlusion and ABI the 6MWT, according to some implementations.

FIG. 9 shows graphs indicating measured and predicted ankle-brachial index (ABI) and Six-Minute Walk Test (6MWT) Continuous Distance in individual patients in the training set and test set, according to some implementations.

FIGS. 10A-10E are graphs showing the performance of the exemplary system and the relationship between predictor variables (e.g., reperfusion slope, Ankle-Brachial Index, walking distance, reperfusion index, percentage of predicted walking distance) for various domains (e.g., distance, speed, stair climbing) during vascular occlusion test (VOT) and Six-Minute Walk Test (6MWT), according to some implementations. FIG. 10A is a diagram a multi-fold leave-one-out cross-validation process for the exemplary system. FIG. 10B shows graphs demonstrating the relationship between Reperfusion Index with Ankle-Brachial Index, Six-Minute Walk Test (6MWT) total distance, and percentage of the predicted 6MWT distance (% 6MWTd). FIG. 10C shows graphs demonstrating the relationship between reperfusion slope with Ankle-Brachial Index, Six-Minute Walk Test (6MWT) total distance, and percentage of the predicted 6MWT distance (% 6MWTd). FIG. 10D shows graphs demonstrating the WIQ domain scores for each domain (e.g., distance, speed, stair climbing) using predictor variables (RI, ABI, reperfusion slope). FIG. 10E shows graphs demonstrating the measured and predicted values of Ankle-Brachial Index (ABI) and percentage of the predicted 6MWT distance (% 6MWTd).

FIGS. 11A-11D shows results for a limb exercise study using assessment based on the exemplary system and method. FIG. 11A shows the limb exercise. FIG. 11B shows results for a vascular occlusion test (VOT) and 6-minute walk test (6MWT) were administered before and after the intervention. FIG. 11C shows results of reperfusion Index (measured with NIRS during the VOT) increased in patients that performed 4 weeks of muscular stretching. FIG. 11D shows results for patients in which the amplitude of the muscle O2 signal improved after stretching also showed an increase in 6MWT distance.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the figures, a system and methods for the automated assessment of microvascular health in skeletal muscle are shown, according to various implementations. In particular, the system and methods described herein evaluate NIRS signals obtained during a VOT to estimate microvascular health and, in some cases, to detect instances of microvascular dysfunction. Currently, the analysis and interpretation of NIRS data from a VOT requires specific knowledge of the physiology of the microcirculation and skeletal muscle that many users (e.g., hospital and clinic staff) do not possess. This estimation of microvascular health and detection of microvascular dysfunction can be integrated into a complete test procedure, which includes performing a VOT to capture the NIRS signals so that non-expert users can perform quick and accurate microvascular health evaluations. Further, the system and methods described herein standardize the analysis of NIRS data for determining microvascular health, which, as mentioned above, has been a long-standing challenge in the field of microvascular and metabolic health.

### Example System

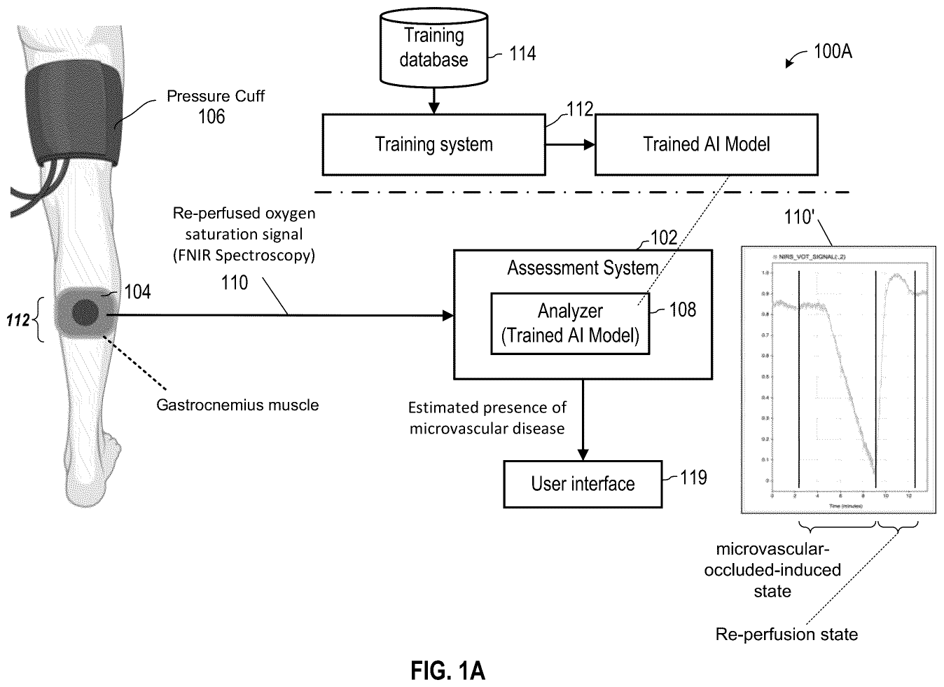

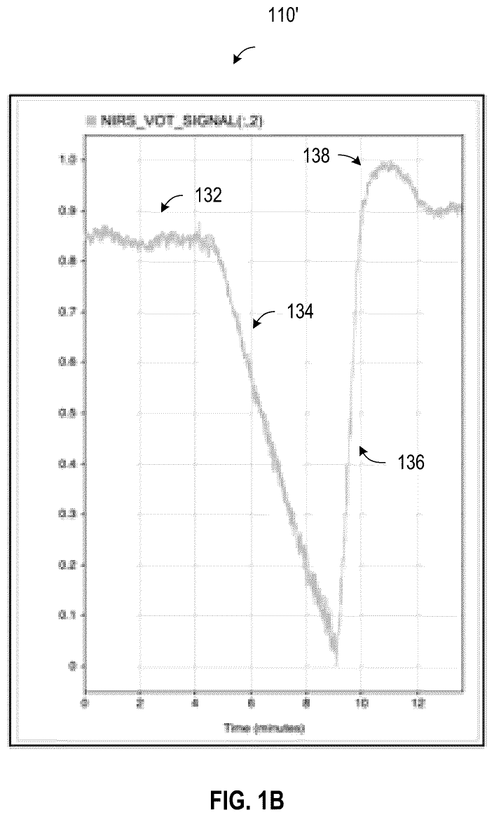

FIG. 1A shows an example system 100A configured to non-invasively use re-perfused oxygen saturation signals from near-infrared spectroscopy signal (NIRS) measurement acquired after a microvascular-occluded-induced state to evaluate microvascular health in a subject in accordance with an illustrative embodiment. In the example of FIG. 1A, the system 100A includes assessment system 102, a spectroscopy device 104, and a pressure cuff 106.

The assessment system 102 employs an analyzer 108 configured with a trained AI model that can (i) obtain a re-perfused oxygen saturation signal 110 acquired via the spectroscopy device 104 and (ii) generate, via a trained AI model or a model derived therefrom, a perfusion index value or score, wherein the trained AI model was trained based on a microvascular function assessment and re-perfused oxygen saturation signal. In some embodiments, the assessment system 102 is a local computing device that may be placed near the spectroscopy device 104. In other embodiments, the assessment system 102 is a remote computing device, e.g., located in cloud infrastructure, that receives the measurement from the spectroscopy device 104 or an edge device connected thereto.

The spectroscopy device 104 (e.g., near-infrared spectroscopy signal) may be placed (e.g., strapped) to the patient at a position 112 at an extremity of the subject (e.g., during a vascular occlusion test (VOT)) downstream to another position having positioned the pressure cuff 106 to cause occlusion of the extremity when inflated. In some embodiments, the position 112 is located at the gastrocnemius muscle. The measurement may be performed when the patient is in a supine position.

The pressure cuff 106 may be inflated for at least 3 minutes, e.g., between 3-5 minutes, to occlude the perfusion of blood in the extremity. The pressure cuff is then deflated to allow reperfusion. The reperfusion is characterized as a perfusion index or score (also referred to as a re-perfusion index or score) by the analyzer 108.

The analyzer 108, operating on the assessment system 102, can receive the re-perfused oxygen saturation signal 110. The analyzer 108 includes a classifier configured to generate an estimation of microvascular health and/or predict the presence of microvascular dysfunction from the oxygen saturation signal 110. The classifier can be configured to output, for a given age patient, the presence of microvascular dysfunction, the non-presence of microvascular dysfunction (e.g., for anomaly detection), a severity score for microvascular dysfunction, an estimated perfusion index or score, an estimate of the likelihood of failing the walking test, an estimated distance to failure in the walking test, or the like. The classifier, as a trained AI model or a model derived therefrom may be generated via a training system 112 configured for supervised or semi-supervised operations of training data stored in a training database 114. The training data may include a re-perfused oxygen saturation signal similarly acquired as signal 110 and labeled with a label for the presence of microvascular dysfunction, label for the non-presence of microvascular dysfunction (e.g., for anomaly detection), label for a severity score for microvascular dysfunction, label for a failing/passing a walking test, or label for a distance to failure in the walking test, or the like.

Example classifiers include a perceptron, Naïve-Bayes, a decision tree, a logistic regression model, a support vector machine (SVM), and the like. It should be appreciated, however, that the specific type of machine learning model is not intended to be limited solely to a classifier. Other suitable types of machine learning models (e.g., deep learning models) are contemplated here.

The assessment system 102 may provide a local user interface or operate with a remote user interface 119 that can show the output of the assessment system 102, e.g., via a doctor, clinician, or patient portal.

Machine Learning. In addition to the machine learning features described above, the analysis system can be implemented using one or more artificial intelligence and machine learning operations. The term “artificial intelligence” can include any technique that enables one or more computing devices or computing systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target) during training with a labeled data set (or dataset). In an unsupervised learning model, the algorithm discovers patterns among data. In a semi-supervised model, the model learns a function that maps an input (also known as a feature or features) to an output (also known as a target) during training with both labeled and unlabeled data.

An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers, such as an input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similarly to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., an error such as L1 or L2 loss), during training. This disclosure contemplates that any algorithm that finds the minimum of the cost function can be used. LR classifiers are known in the art and are therefore not described in further detail herein.

A Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

### Example Re-Perfused Oxygen Saturation Signal

FIG. 1B shows the re-perfused oxygen saturation signal 110 (shown as 110′) of FIG. 1A as acquired by an FNIR device. For this measurement, the NIRS device was placed below the pressure cuff to induce occlusion during all parts of a vascular occlusion test. As shown in graph 100B, a baseline period 132 of signal 110′ corresponds to a measurement of oxygen saturation when the subject is at rest. The occlusion of blood flow eliminates oxygen delivery and causes a sharp decrease in oxygen saturation, as indicated by an occlusion period 134 of signal 110′. Upon release of the pressure cuff, oxygen saturation increases rapidly, as indicated by a reperfusion period 136. Oxygen saturation quickly returns to the baseline value and then briefly exceeds the baseline during a hyperemic period 138 before finally returning to the baseline value.

The rate of decline in saturation after occlusion (e.g., during occlusion period 134) is an indicator of oxygen extraction and utilization by the muscle. The rapid increase in oxygen saturation that occurs upon release of the occlusion (e.g., during reperfusion period 136) is an indicator of microvascular reperfusion (e.g., the influx of oxygenated blood through the microcirculation). The hyperemic/overshoot period (e.g., hyperemic period 138) is an indicator of the reactivity of the resistance vasculature that is responsible for the distribution of blood flow through the muscle. By extension, the NIRS signal produced can be used as a biomarker for microvascular function and metabolic capacity of the limb musculature.

The VOT is a standardized protocol for assessing oxygenation in skeletal muscle. The VOT is typically executed in approximately 20 minutes. Muscle oxygenation is assessed at a baseline (e.g., without occlusion) during a first time period and during a second time period of complete occlusion of blood flow to an extremity. Typically, occlusion is induced by the inflation of a pressure cuff. Each “time period” is typically about five minutes; however, the present disclosure is not limiting in this regard. Following the first and second time periods, muscle oxygenation is assessed during a third time period (e.g., five minutes) of reperfusion of the extremity after the occlusion is released. In some implementations, the VOT is performed with the subject supine; however, the present disclosure is not intended to be limited in this regard.

### Example Method of Training an AI Model for Microvascular Function Assessment

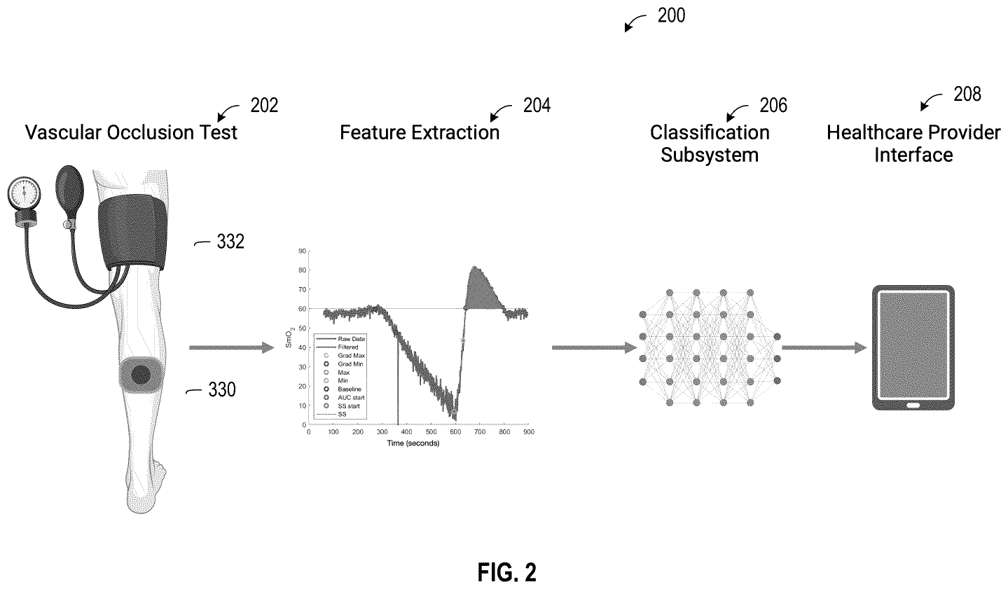

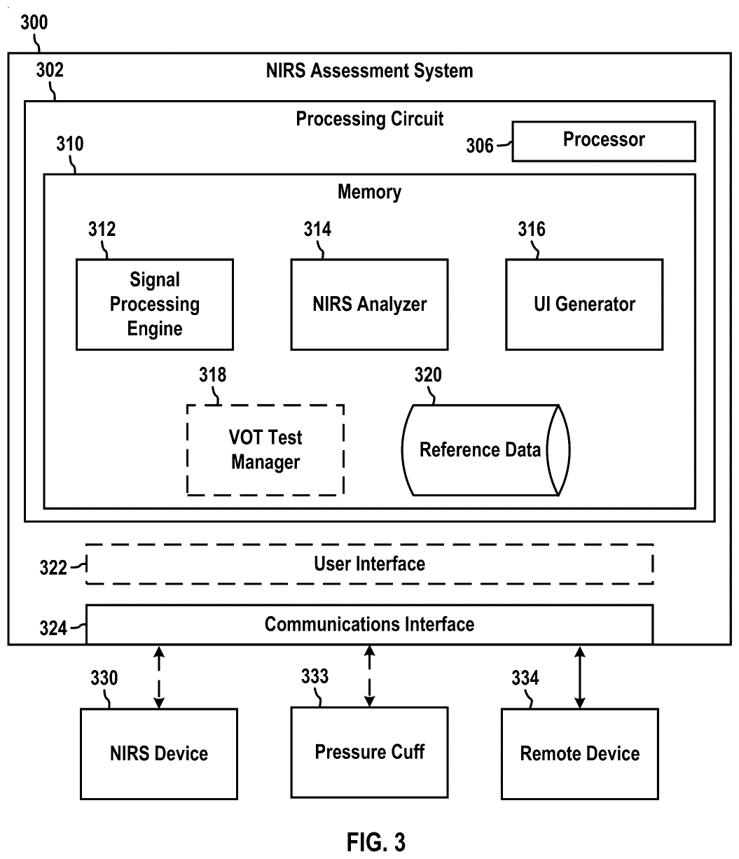

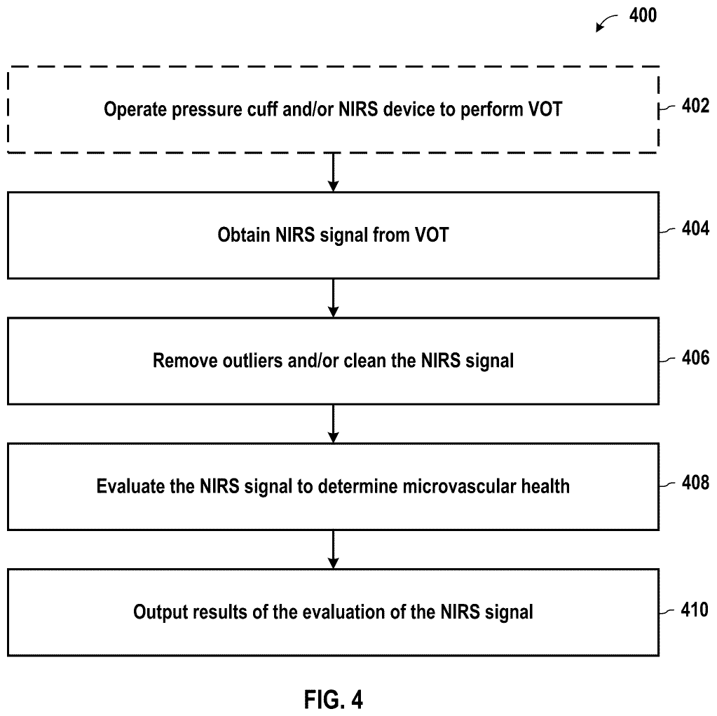

FIG. 2 shows an example method of generating the trained AI model, including the signal collection and processing pipeline 200 for evaluating microvascular function in a subject, according to some implementations. FIGS. 3 and 4 provide a further description of the process shown in FIG. 2. It should be appreciated that certain steps shown in pipeline 200 may be optional and, in some implementations, pipeline 200 additional or fewer steps. It will also be appreciated that the order of steps shown in FIG. 2 is not intended to be limiting.

In FIG. 2, at step 202, a vascular occlusion operation is performed (e.g., a vascular occlusion test) on a subject. In the example shown, the vascular occlusion operation is being conducted on the subject's leg; however, it should be appreciated that a VOT can be performed on any extremity. In FIG. 3, NIRS device 330 (as an example of a spectroscopy device 104 in FIG. 1A) is positioned below (e.g., distally from) a pressure cuff 332 (shown as 106 in FIG. 1A) during the vascular occlusion operation. The NIRS device 330 is generally configured to record a NIRS signal (e.g., based on the relative absorption of light in the near-infrared range) indicative of skeletal muscle oxygen saturation, and pressure cuff 332 is configured to selectively induce occlusion of blood flow in the leg.

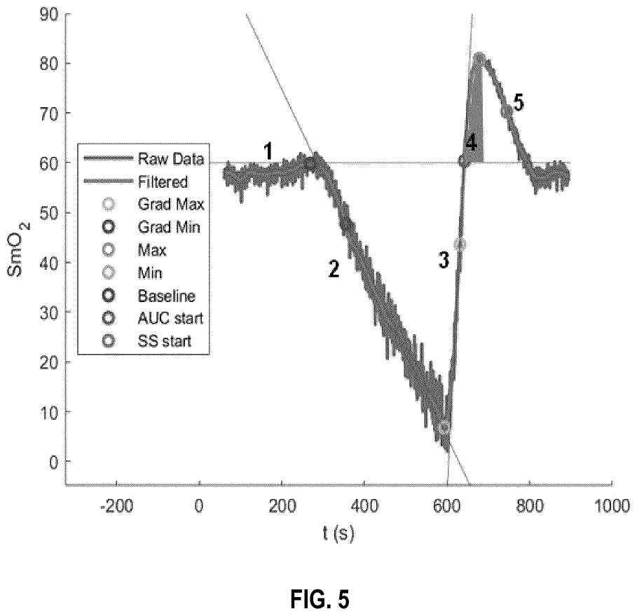

Once an NIRS signal is recorded (e.g., over the course of the vascular occlusion operation), it may optionally be preprocessed, e.g., through filtering, to reduce noise, remove outliers, and the like. Then, or in cases where the NIRS signal is not preprocessed after recording the signal, features are extracted from the signal at step 204. “Features,” as used herein, generally refers to physiological features that are the indicators of microvascular function. These physiological features includes include: (i) a rate of oxygen utilization detected by the rate of oxygen desaturation of the muscle during the period of occlusion; (ii) a rate of microvascular reperfusion detected by the rate of oxygen restoration of the muscle measured immediately upon release of the occlusion; (iii) the hyperemic (overshoot) response of the muscle microvasculature detected by the area under the oxygen saturation curve that exceeds the baseline level (e.g., measured before the initiation of occlusion); (iv) recovery response of the muscle microvasculature as detected by the rate of return oxygen saturation to baseline after the occlusion; and (v) the overall oxygenation of the muscle throughout the test as determined by the area under the curve during baseline, occlusion, and post-occlusion. Further discussion of the physiological features, including the rationale for selecting these specific features, is provided below with respect to FIGS. 5-8C.

At step 206, the extracted features are provided as inputs to a predictive model that estimates microvascular function and, in some implementations, identifies instances of microvascular dysfunction. As described in greater detail below, the predictive model may be machine learning-based. Specifically, in some such implementations, the predictive model is a classifier. As will be appreciated, a classifier is a machine learning model that predicts a class or “class label” for input data. In this case, the classifier may predict whether the microvascular health of the subject is normal (e.g., a first class) or indicative of a known disorder/dysfunction (e.g., one of a plurality of second classes). Example classifiers include a perceptron, Naïve-Bayes, a decision tree, a logistic regression model, a support vector machine (SVM), and the like. It should be appreciated, however, that the specific type of machine learning model is not intended to be limited solely to a classifier. Other suitable types of machine learning models (e.g., deep learning models) are contemplated here.

In other implementations, the predictive model is configured to compare the extracted features to a set of standardized values generated from known healthy subjects. In some such implementations, the “known-healthy subjects” include a plurality of persons who are known not to be affected by any sort of microvascular dysfunction. Often, the standardized values are obtained from known healthy subjects that are of an age range corresponding to the subject being evaluated. In yet other implementations, the predictive model is configured to compare the NIRS signal directly (e.g., with or without feature extraction) with a reference signal generated from known healthy subjects. In some such implementations, NIRS signals may be collected from a plurality of known healthy subjects during a VOT and used to generate an “optimized” or “ideal” reference signal, to which the subsequent NIRS signals from other patients can be compared.

After evaluating the NIRS signal and/or extracted features, at step 208, an indication of microvascular health is provided via a user interface (shown as 119 in FIG. 1A). In some implementations, the indication of microvascular health includes a standardized value for each of the physiological features and/or a “total health” value. As described herein, the standardized values for physiological features and/or “total health” values refer to values that are calculated from the analysis of the NIRS signal to indicate microvascular health on a standardized scale. In other words, the standardized values and/or “total health” values are easy-to-understand values that indicate microvascular health to a user. For example, the “total health” value may be a value out of 100 (e.g., expressed as a percentage), indicating a closeness to the “ideal” of the subject's microvascular health. In some implementations, the indication includes graphs, charts, or other graphical elements that clearly and unambiguously indicate to a user microvascular health. In some implementations, the indication includes alerts or notifications if microvascular dysfunction is detected, as discussed in greater detail below.

FIG. 3 shows a NIRS assessment system 300 is shown, according to some implementations. Generally, system 300 is a computing device configured to obtain and analyze NIRS signals to evaluate microvascular health in various subjects. As alluded to above, system 300 may output easy-to-understand indicators of microvascular health and, in some instances, can be configured to predict certain types of microvascular dysfunction in subjects that produce abnormal NIRS data. In some implementations, as discussed below, system 300 can automate the collection of NIRS signals as well by controlling various external devices to perform a VOT and record NIRS signals.

System 300 is shown to include a processing circuit 302 that includes a processor 306 and a memory 310. Processor 306 can be a general-purpose processor, an application-specific integrated circuit (ASIC), one or more field programmable gate arrays (FPGAs), a group of processing components (e.g., a central processing unit (CPU)), or other suitable electronic processing structures. In some implementations, processor 306 is configured to execute program code stored on memory 310 to cause system 300 to perform one or more operations, as described below in greater detail. It will be appreciated that, in implementations where system 300 is part of another computing device, the components of system 300 may be shared with, or the same as, the host device. For example, if system 300 is implemented via a server, then system 300 may utilize the processing circuit, processor(s), and/or memory of the server to perform the functions described herein.

Memory 310 can include one or more devices (e.g., memory units, memory devices, storage devices, etc.) for storing data and/or computer code for completing and/or facilitating the various processes described in the present disclosure. In some implementations, memory 310 includes tangible (e.g., non-transitory), computer-readable media that store code or instructions executable by processor 306. Tangible, computer-readable media refers to any physical media that is capable of providing data that causes system 300 to operate in a particular fashion. Examples of tangible, computer-readable media may include, but are not limited to, volatile media, non-volatile media, removable media, and non-removable media implemented in any method or technology for storage of information such as computer-readable instructions, data structures, program modules, or other data. Accordingly, memory 310 can include random access memory (RAM), read-only memory (ROM), erasable programmable read-only memory (EPROM), electronically erasable programmable read-only memory (EEPROM), hard drive storage, temporary storage, non-volatile memory, flash memory, optical memory, or any other suitable memory for storing software objects and/or computer instructions. Memory 310 can include database components, object code components, script components, or any other type of information structure to support the various activities and information structures described in the present disclosure. Memory 310 can be communicably connected to processor 306, such as via processing circuit 302, and can include computer code for executing (e.g., by processor 306) one or more processes described herein.

While shown as individual components, it will be appreciated that processor 306 and/or memory 310 can be implemented using a variety of different types and quantities of processors and memory. For example, processor 306 may represent a single processing device or multiple processing devices. Similarly, memory 310 may represent a single memory device or multiple memory devices. Additionally, in some implementations, system 300 may be implemented within a single computing device (e.g., one server, one housing, etc.). In other implementations, system 300 may be distributed across multiple servers or computers (e.g., that can exist in distributed locations). For example, system 300 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers.

Memory 310 is shown to include a signal processing engine 312 configured to obtain and optionally preprocess NIRS signals. Generally, NIRS signals are recorded by NIRS device 330 (e.g., a spectrometer), which, as known in the art, is a device configured to measure muscle oxygenation using near-infrared light. In some implementations, signal processing engine 312 receives NIRS signal data directly from NIRS device 330, e.g., during or after a VOT For example, system 300 may be communicably coupled to NIRS device 330 so that raw NIRS signals are received directly by signal processing engine 312. In other implementations, signal processing engine 312 receives NIRS signal data from a remote device 334, such as another computing device, a server, etc. For example, NIRS device 330 may alternatively be communicably coupled to remote device 334 for recording NIRS signal data, which is then transmitted or uploaded to system 300. In yet other implementations, signal processing engine 312 is configured to retrieve pre-recorded NIRS signals from remote device 334 and/or a remote database.

As mentioned, in some implementations, signal processing engine 312 can optionally preprocess NIRS signals to place the NIRS signals in better condition for further analysis/processing. Generally, signal processing engine 312 can implement a variety of known signal-processing techniques. For example, in some cases, signal processing engine 312 converts analog signals into digital signals, e.g., using an analog-to-digital converter (ADC). In some implementations, signal processing engine 312 filters NIRS signals using one or more analog and/or digital filters. For example, analog and/or digital filters can be used to remove noise, outliers, and the like, to “clean” the NIRS signal data. In some implementations, signal processing engine 312 is configured to denoise the NIRS signal and/or remove outliers via smoothing. In this regard, to remove outliers and improve the number of useful samples, signal processing engine 312 may include and/or implement an adaptive smoothing filter, such as a seven-tap outlier detection filter. In some such implementations, if a raw signal varies significantly from a median of measurements in a window, the measurement may be replaced by the median or simply removed. In some implementations, a moving average filter is applied to smooth the NIRS signal. Specifically, within a moving average, the number of data points to include in the average will change the smoothing of the NIRS signal.

Memory 310 also includes a NIRS analyzer 314 (as an example of an analyzer 108 previously shown in FIG. 1A) that is configured to evaluate microvascular health based on obtained NIRS signals. In particular, NIRS analyzer 314 generates an estimation of microvascular health and/or can predict the presence of microvascular dysfunction. In some implementations, NIRS analyzer 314 may first extract physiological features indicative of microvascular function from an obtained NIRS signal. As mentioned above, these physiological features are generally predefined and can include, but are not limited to, a rate of oxygen utilization during a period of occlusion, a rate of microvascular reperfusion upon release of occlusion, a hyperemic response of muscle microvasculature, a rate of return of oxygen saturation to baseline after occlusion, and overall oxygenation of muscle throughout the VOT.

At least some of the physiological features are extracted by evaluating the NIRS signal and performing calculations based on the NIRS signal data. For example, the hyperemic (overshoot) response of the muscle microvasculature may be calculated based on the area under the oxygen saturation curve in the NIRS signal exceeding a baseline level. As another example, the rate of oxygen desaturation during the occlusion phase is calculated by determining the highest oxygen saturation level before the occlusion, the lowest oxygen saturation level during the occlusion, the level of oxygen saturation at the midpoint between high and low, then the rate of change around the midpoint. In some implementations, certain features are extracted only after smoothing the NIRS signal data (e.g., by the signal processing engine 312).

After extraction, the physiological features are provided as input to a predictive model of NIRS analyzer 314. The predictive model is generally configured to output an estimation of microvascular health and/or a prediction of microvascular dysfunction, e.g., in the form of easy-to-understand values. Alternatively, NIRS analyzer 314 may interpret the output of the predictive model to provide an intuitive indication of microvascular health. In some implementations, the predictive model is or includes a classifier or other suitable machine learning-based model (e.g., a support vector machine (SVM) or another deep learning model). A classifier, as mentioned above, predicts a class that best fits input data (e.g., the physiological features). In other words, in some implementations, the predictive model of NIRS analyzer 314 classifies input data (e.g., physiological features) into a class associated with “normal” or “healthy” microvascular function and/or into one of a plurality of different classes each associated with a specific microvascular dysfunction. As an example, a significant reduction in the rate of oxygen desaturation may be an indicator of loss of oxygen diffusivity (loss of capillaries) or a decrease in the ability of the muscle to extract and consume oxygen. As another example, a significant reduction in the rate of microvascular reperfusion may also an indicator of a loss of capillaries and also an indicator of microendothelial function (the reactivity of the endothelium lining the microvasculature is impaired). NIRS analyzer 314 can, in this regard, be configured to determine a suspected microvascular dysfunction (e.g., loss of oxygen diffusivity).

In implementations where NIRS analyzer 314 is or includes a machine learning model for evaluating NIRS signals, the machine learning model (e.g., a classifier) can be trained using training data obtained from known healthy subjects. In some cases, multiple versions of a machine learning model are trained using data from known healthy subjects of different demographics. For example, the machine learning model may be trained using training data from a specific age range of known healthy subjects, such that the resulting trained model can evaluate NIRS data from a similarly-aged subject of interest. Said training data—or, more generally, the NIRS signal data obtained from known-healthy subjects—may be retained in a reference database 320 (shown as 117 in FIG. 1A). In this regard, the machine learning model (e.g., classifier) of NIRS analyzer 314 may be trained using supervised and/or semi-supervised techniques, as known to those in the art.

In other implementations, rather than utilizing or including a classifier, NIRS analyzer 314 includes another type of predictive model suitable for estimating microvascular health/dysfunction. For example, the predictive model may compare the extracted physiological features to a set of standardized values generated from known healthy subjects. These standardized values may be stored in reference database 320 or may be retrieved from a remote database. In some such implementations, the “known-healthy subjects” may be of an age range corresponding to the subject of interest, such that the physiological features obtained from the “known-healthy subjects” can act as a baseline for determining the microvascular health of the subject of interest. In some such implementations, deviations between any of the extracted physiological features and a corresponding one of the set of standardized values indicate microvascular dysfunction. In yet other implementations, the predictive model of NIRS analyzer 314 generates “optimized” or “ideal” NIRS signal data, e.g., by simulating the results of a VOT performed on a known-healthy subject (e.g., a parameterized computer model) using the data in reference database 320, and/or NIRS analyzer 314 references a previously captured NIRS signal from a known-healthy subject, which is then compared to the NIRS signal of the subject of interest to identify deviations indicative of microvascular dysfunction.

Memory 310 is also shown to include a user interface generator 316 configured to generate graphical user interfaces (GUIs) based on the analysis of NIRS signals, e.g., by NIRS analyzer 314. In particular, user interface generator 316 can generate a GUI or GUIs that intuitively display results of the evaluation of NIRS signals, such that non-expert users can easily understand them. For example, user interface generator 316 can generate a GUI that displays standardized or “normalized” values associated with each of the extracted physiological features and/or that provides a “total score” indicative of microvascular health. As another example, the GUI(s) generated by user interface generator 316 may include graphs, charts, or other graphical elements that present an indication of microvascular health and/or dysfunction graphically. In some implementations, the GUI indicates a deviation of a specific physiological feature or the estimation of the subject's overall microvascular health from a mean or an expected (e.g., “healthy”) value. In some implementations, user interface generator 316 is configured to generate alerts or notifications if microvascular dysfunction is predicted (e.g., by NIRS analyzer 314). For example, if the subject's NIRS signal indicates some form of microvascular dysfunction, user interface generator 316 may generate an alert that indicates the microvascular dysfunction and/or a deviation in the associated features of the NIRS signal.

In this regard, system 300 can optionally include an integrated user interface 322 (as an example of a user interface 119 shown in FIG. 1A) for presenting the GUIs generated by user interface generator 316. Generally, user interface 322 includes one or more components that allow a user to interact with system 300. In some implementations, user interface 322 includes a display or “screen,” such as an LED or LCD screen, for displaying GUIs. In some implementations, user interface 322 includes one or more user input devices, e.g., to receive user inputs, such as a keyboard, a mouse, etc. In some implementations, user interface 322 includes other audio and/or visual components, such as lights (e.g., LEDs), a speaker, and the like. For example, user interface 322 may include a speaker for providing an audible alert if microvascular dysfunction is detected.

In some implementations, memory 310 includes a VOT test manager 318 configured to operate NIRS device 330 and/or pressure cuff 333 to perform an automated VOT. In particular, VOT test manager 318 may provide control signals to pressure cuff 333 to cause pressure cuff 333 to selectively inflate/deflate according to a VOT test procedure. In conjunction with controlling pressure cuff 333, or separately, VOT test manager 318 may operate NIRS device 330 to collect NIRS signal data. For example, VOT test manager 318 may initiate a VOT by recording signals via NIRS device 330 for a first time period, then may activate (e.g., inflate) pressure cuff 333 at the end of the first time period to initiate occlusion. Then, at the end of a second time period, VOT test manager 318 may deactivate (e.g., deflate) pressure cuff 333 to release occlusion, all while NIRS device 330 is recording NIRS data.

As described herein, pressure cuff 333 generally refers to any suitable medical device that can cause occlusion (e.g., blocking of blood flow) in an extremity (e.g., an arm or leg). In some implementations, pressure cuff 333 is inflated/deflated by increasing/decreasing an internal air or fluid pressure, e.g., using a pump. However, it should be appreciated that the specific type and/or construction of pressure cuff 333 is not intended to be limiting herein; rather, pressure cuff 333 may be any suitable device for selectively occluding an extremity and, in some cases, is controllable by VOT test manager 318 as discussed above.

System 300 is also shown to include a communications interface 324 that facilitates communications between system 300 and any external components or devices, including NIRS device 330, pressure cuff 333, and/or remote device 334. In other words, communications interface 324 can provide a means for transmitting data to and/or receiving data from any of NIRS device 330, pressure cuff 333, and/or a remote device 334. Additionally, or alternatively, communications interface 324 can transmit control signals and/or power to certain external devices (e.g., pressure cuff 333). Accordingly, communications interface 324 can be or can include a wired or wireless communications interface (e.g., jacks, antennas, transmitters, receivers, transceivers, wire terminals, etc.) for conducting data communications or a combination of wired and wireless communication interfaces. In some implementations, communications via communications interface 324 are direct (e.g., local wired or wireless communications) or via a network (e.g., a WAN, the Internet, a cellular network, etc.). For example, communications interface 324 may include one or more Ethernet ports for communicably coupling system 300 to a network (e.g., the Internet). In another example, communications interface 324 can include a Wi-Fi transceiver for communicating via a wireless communications network.

Remote device 334 generally refers to any external computing device that can communicate with system 300. Examples of remote device 334 include a server, a desktop or laptop computer, a workstation, a tablet, a smartphone, and the like. In some implementations, remote device 334 can receive and/or provide NIRS signal data. In some implementations, remote device 334 receives the results of evaluating the NIRS signal data, e.g., by NIRS analyzer 314. In some implementations, remote device 334 can act as a remote display for viewing the results of said evaluation, e.g., as opposed to system 300 including a dedicated user interface. For example, system 300 may be configured to locally evaluate NIRS signals, e.g., to determine microvascular health, and then may transmit the results of the evaluation to remote device 334 for viewing, storage, etc.

Referring now to FIG. 4, a flow chart of a process 400 for estimating microvascular function in a subject using NIRS data is shown, according to some implementations. In some implementations, process 400 is implemented by system 300, as described above. It will be appreciated that certain steps of process 400 may be optional and, in some implementations, process 400 may be implemented using less than all of the steps. It will also be appreciated that the order of steps shown in FIG. 4 is not intended to be limiting.

At step 402, a pressure cuff and/or NIRS device are optionally operated to perform a VOT. In particular, a signal from the NIRS device may be recorded to capture a NIRS signal associated with the VOT. In conjunction, the pressure cuff may be automatically inflated/deflated or otherwise controlled to induce and subsequently release occlusion of an extremity in which microvascular health is being evaluated. In this regard, the VOT may be automated, e.g., to significantly reduce the chances of operator error and to allow non-expert users to perform a VOT. Generally, in implementations where step 402 is performed, the pressure cuff and/or NIRS device are operated in accordance with a standardized VOT protocol. For example, the NIRS device may record muscle oxygenation at a baseline (e.g., without occlusion) during a first time period and during a second time period of complete occlusion of blood flow to an extremity. At the end of the first time period, the pressure cuff may be activated to induce occlusion. Following the first and second time periods, muscle oxygenation is assessed during a third time period (e.g., five minutes) of reperfusion of the extremity after the occlusion is released.

At step 404, a NIRS signal is obtained from the VOT. In implementations where step 402 is performed, the NIRS signal may be received and/or recorded directly from an NIRS device. In other implementations, the NIRS signal may be separately recorded using a suitable NIRS device. In some such implementations, the NIRS signal may be retrieved at step 404 (e.g., from a database) or may be received from a remote device (e.g., received by system 300 from remote device 334, such as a controller of the NIRS device).

At step 406, the NIRS signal is preprocessed to remove outliers and/or otherwise clean the signal. As mentioned above, preprocessing can include any number of different functions. In some implementations, preprocessing includes using analog and/or digital filters to remove outliers and improve the number of useful samples. In some such implementations, the analog and/or digital filters include a seven-tap outlier detection filter. In some such implementations, if a raw signal varies significantly from a median of measurements in a window, the measurement may be replaced by the median or simply removed. In some implementations, preprocessing includes converting an analog signal into a digital signal, e.g., using an ADC.

At step 408, the NIRS signal is evaluated to determine microvascular health. As discussed above, evaluating the NIRS signal generally includes extracting physiological features indicative of microvascular function. Then, the extracted physiological features are provided as input to a predictive model (e.g., a classifier). The predictive model outputs an indication of microvascular health, e.g., in the form of a value associated with each physiological feature, in the form of a total “microvascular health” value, and/or in another suitable but easy-to-understand format. In some implementations, the output of the predictive model is further analyzed to determine the presence of microvascular dysfunction. For example, the NIRS signal and/or extracted features may be compared to values associated with known healthy subjects to identify deviations indicative of microvascular dysfunction. In another example, the predictive model can output a classification based on the physiological features, e.g., classifying the NIRS signal data as indicative of “normal” microvascular health or “microvascular dysfunction.”

At step 410, the results of the NIRS signal evaluation (e.g., the output of the predictive model) are returned. In some implementations, this includes presenting an indication of microvascular health, microvascular dysfunction, and/or the analysis of each physiological feature via a user interface. For example, microvascular health may be indicated by one or more intuitive values (e.g., a percentage) and/or graphical elements (e.g., charts, graphs, etc.). In some implementations, a prediction of microvascular dysfunction can be presented as an alert. For example, if microvascular dysfunction is detected, a user may be alerted via a sound, a notification displayed on a screen, etc. In some such implementations, the alert may include an indication of the specific microvascular dysfunction or abnormality detected.

### Experimental Results and Additional Examples

Several studies were conducted to develop and evaluate an analysis system in which NIRS measurements are obtained during the VOT, and in which the analysis of the NIRS data obtained during the test is automated and coupled to standardized readouts in a manner that is analogous to the standardization of blood pressure readouts. By coupling automated analysis to the standardized VOT, assessment of the microvascular function and aerobic metabolism of skeletal muscle can be accomplished independent of the domain expertise provided by cardiologists or skeletal muscle physiologists.

The NIRS signal provides a measurement of muscle oxygenation by determining hemoglobin saturation with oxygen. The VOT provides a standardized protocol for assessing muscle oxygenation that is executed in approximately 20 minutes. Muscle oxygenation is assessed during 5 minutes of baseline, then during 5 minutes of complete occlusion of blood flow to the limb (induced by an inflation of a pressure cuff), followed by 5 minutes of reperfusion of the limb after the occlusion is released. The NIRS device is placed below the cuff during all parts of the VOT.

Correlation Between NIRS Features and Clinical Parameters. FIGS. 5-8C shows the experimental results of the investigation of the first study. Specifically, the features from NIRS signals during the VOT were compared with the Ankle-Brachial Index (ABI) and walking distances measured in a 6-minute walk test.

Participants: Twenty-four PAD patients between the ages of 40-90 years with an ABI≤0.90 in either leg were included in this experiment. Patients with lower limb amputation, critical limb ischemia, kidney disease, or revascularization within the previous three months were excluded.

Measurement of SmO₂ during the VOT: Patients rested quietly in a supine position. Systolic and diastolic blood pressure were measured using a brachial sphygmomanometer. The pulse rate was recorded. Muscle oxygen saturation (SmO₂) was measured during a VOT using a continuous-wave NIRS spectrometer. The NIRS probe was placed on the middle of the belly of the lateral head of the gastrocnemius of the leg most affected by PAD. A thigh cuff was positioned on the thigh just above the knee. Baseline SmO2 was collected for five minutes prior to cuff inflation. At the end of this baseline period, the cuff was inflated to a pressure that was 70-80 mmHg above the systolic blood pressure of the participant to completely occlude flow to the lower limb. Occlusion was verified by the absence of a pulse in the tibial artery. Occlusion was maintained for five minutes, and the cuff was then deflated. NIRS recording of SmO₂ was continuous during baseline, occlusion, and for five minutes post-occlusion, as shown in FIG. 5.

Six-Minute Walk Test (6MWT): Following the VOT and a 20-minute rest period, participants completed a 6MWT according to the American Thoracic Society guidelines. The 6MWT is a standardized test used to assess walking performance, as known to those of ordinary skill in the art. Participants were read validated, standardized directions for the test. The participant was instructed to notify the researcher when the onset of leg pain occurred during the 6MWT. Participants walked for six minutes, back and forth, over a distance of 30 meters marked by two cones, with a goal of covering as much distance as possible in the allotted time. If the participant needed to stop and rest during the test, the distance covered until the first stop was recorded as continuous walking distance. At the conclusion of the six-minute period, the researcher asked the participant to stop walking, and total walking distance (TWD) was recorded in meters as the sum of the number of full and partial laps. Pain-free walk distance (PFWD) was recorded in meters as the distance before the onset of claudication pain.

Analysis of NIRS VOT signal: SmO₂ signals (y) were low-pass filtered (2.5 MHz cutoff, 95% steepness) to remove noise. The first (y′) and second (y″) numerical derivatives were calculated and filtered the same way. The first 60 seconds and final five seconds of the filtered signal were truncated to remove filtering artifacts. Using maxima and minima of y, y,′ and y″, specific time points corresponding to specific features were identified. Average slopes were obtained by the linear fit between these points or, in the case of the area under the curve, numerical integration. The features extracted from the NIRS VOT signal were: i) average SmO₂ at baseline, ii) the average slope during occlusion (occlusion slope, ΔSmO₂/s), iii) the average slope from cuff release until reaching baseline (reperfusion slope, ΔSmO2/s), iv) post-occlusion area under the curve, and v) the average slope of the return to baseline from the hyperemic peak, as shown in FIG. 5.

Statistical Analysis: Correlations between features extracted from the NIRS/VOT signal and clinical outcome variables (ABI and 6MWT distances) were evaluated by linear regression, with the generation of R and R². Significance was set at α≤0.05.

Results: Characteristics for 24 patients are shown in Table 1. There was an even distribution of men and women. The mean age was 71.3 years. The mean ABI was 0.61. Mean total and continuous walking distances on the 6MWT were 299.6 meters and 284.5 meters, respectively. 74.4% of patients had hypertension, and 33.3% had Type II Diabetes.

TABLE 1
Patient Characteristics

| Gender | Female: 50.00% | Male: 50.00% |
|---|---|---|
| Hypertension | Yes: 74.36% | No: 25.64% |
| Diabetes | Yes: 33.33% | No: 66.67% |
| Smoking | Yes: 25.64% | No: 74.36% |
| Most Affected Leg | Right: 52.50% | Left: 47.50% |
| Height (cm) | 169.30 ± 9.34 | |
| Weight (kilograms) | 79.63 ± 19.44 | |
| Waist circumference (cm) | 106.22 ± 10.46 | |
| Left lower leg circ. (cm) | 35.08 ± 4.07 | |
| Right lower leg circ. (cm) | 34.64 ± 3.03 | |
| MAP | 95.62 ± 13.20 | |
| Pulse Pressure | 64.93 ± 16.55 | |
| Resting Diastolic Pressure | 74.26 ± 9.76 | |
| Resting Systolic Pressure | 137.72 ± 20.28 | |
| Distance calcaneus boney | 35.61 ± 5.04 | |
| landmark to NIRS (cm) | | |

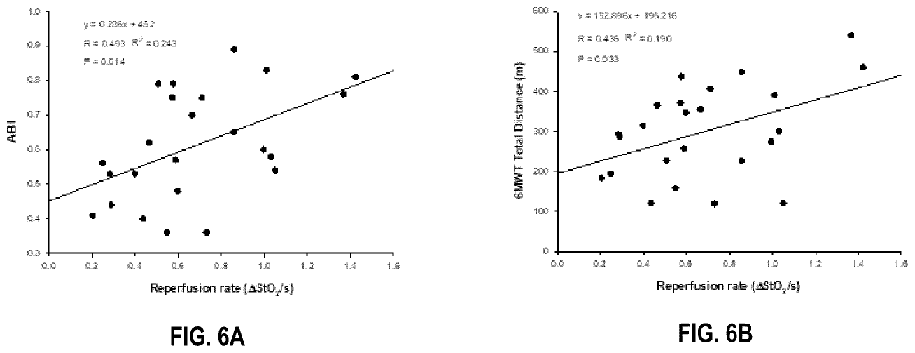

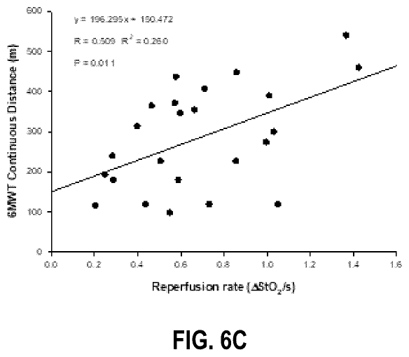

Regression analysis demonstrated a significant relationship between ABI and the rate of increase in SmO₂ (reperfusion rate) in the calf muscle following the release of the five-min test occlusion (P=0.01), with an R-value of 0.49 and an R² value of 0.24, as shown in FIGS. 6A-6C. Similarly, a significant relationship was detected between total (P=0.03) and continuous (P=0.01) walking distances measured during the 6MWT and the rate of increase of SmO₂ in the calf following release of occlusion, with R² values of 0.19 and 0.26, respectively.

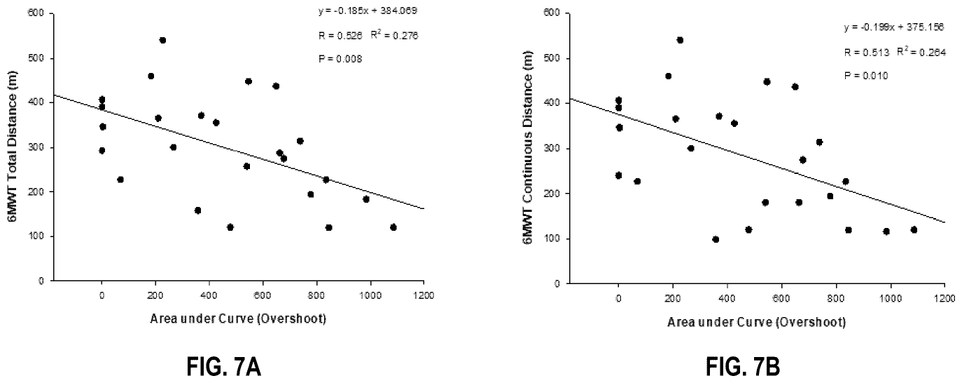

FIGS. 7A and 7B show that the correlation between continuous walking distance in the 6MWT and the area under the post occlusion hyperemic curve (shown as area (4) in FIG. 5) was highly significant (P=0.008, R²=0.276), as was the correlation between total walking distance in the 6MWT and the area under the post occlusion hyperemic curve (P=0.01, R²=0.264). The correlation between ABI and the area under the post-occlusion hyperemic curve was nearly significant, with a P-value of 0.06 (data not shown).

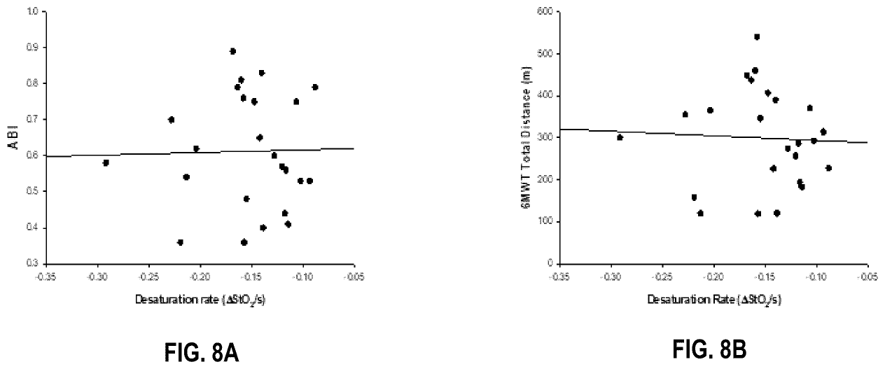

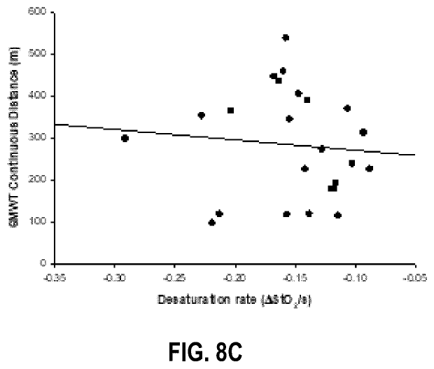

Regression analysis further revealed that there was no significant correlation between the rate of oxygen desaturation in the calf (ΔSmO₂/s) during given minutes of occlusion and ABI. Similarly, no correlation was found between the rate of oxygen desaturation in the calf muscles during five minutes of occlusion and either total or continuous walking distance during the 6MWT, as shown in FIGS. 8A-8C.

Discussion: The objective of the above-discussed experiments was to extract features from a NIRS signal that included measurement of SmO₂ at baseline, during five minutes of occlusion of the calf muscles, and during post-ischemic recovery (often referred to as a VOT) in patients with PAD. Key physiological features were extracted from the VOT signal, and the features were compared with conventional ABI. The established measurements (continuous walking distance and total walking distance) were obtained from the 6-minute walk test (6MWT). It was hypothesized that physiological features extracted from the NIRS signal would correlate with established clinical measurements of atherosclerosis in the leg arteries (ABI) and measurements of walking function (6MWT distances). NIRS assessment of SmO₂ during the vascular occlusion test provides a reproducible assessment of oxygen delivery through the microcirculation as well as oxygen diffusivity and utilization by muscle. A key finding is that NIRS assessment of SmO₂ during the VOT provides an assessment of microvascular delivery function in the calf muscles of PAD patients that correlates with measures of arterial perfusion (ABI) and walking function (6MWT distances) while also potentially providing an assessment of metabolic function in the calf muscles.

The health of skeletal muscle is an important determinant of disease progression, disability, and death, especially in older patients with declining mobility and in clinical populations that include patients with diabetes. In PAD, the information yielded by NIRS provides an assessment of key pathophysiological determinants of disease progression, specifically oxygen delivery and utilization in muscles of the lower limbs. NIRS profiles in PAD-affected lower extremities are characterized by rapid changes in oxygen saturation, significant hypoxia during exercise, and slow return to baseline oxygen saturation post-exercise. These profiles have a strong inverse correlation with walking capacity, independent of ABI. Numerous clinical studies have utilized NIRS to determine SmO₂ and to estimate muscle oxygen consumption during exercise or occlusion.

The experimental data discussed above indicates a significant correlation between ABI and the rate of reperfusion of the calf muscles following five minutes of occlusion (FIGS. 6A-6C). A significant relationship between reperfusion rate and continuous and total walking distances in the 6MWT was also demonstrated. These findings support the notion that NIRS measurement of SmO₂ during the VOT is a valid assessment of microvascular perfusion and oxygen delivery that is compromised in patients with PAD. Agreement with the standard clinical measure of ABI, as well as walking outcomes, suggests that NIRS assessment during the VOT could be used predictively of functional status that is linked to microvascular dysfunction in addition to macrovascular disease.

### Additional Experiment Set #2

The study also investigated whether NIRS measurement of muscle oxygenation during a standard test of vascular occlusion and post-occlusive hyperemia and the vascular occlusion test (VOT) is predictive of PAD severity as determined by ABI and 6MWT.

Methods: 24 patients diagnosed with PAD were included in this experiment. The mean age of the patients was 71.3 years, including 54% (n=13) males and 46% (n=11) females. The VOT consisted of rest, occlusion, and reperfusion phases, each lasting 5 minutes (15 minutes total). Muscle oxygen saturation levels were recorded at 2 hertz. For every patient, 6 features were extracted from the VOT data using computational methods. The VOT features from 15 patients were used to train function-fitting neural network models to predict ABI and 6MWT Continuous Distance. The models were then used to predict ABI and 6MWT Continuous Distance from the VOT features of 9 test patients not used in the training.

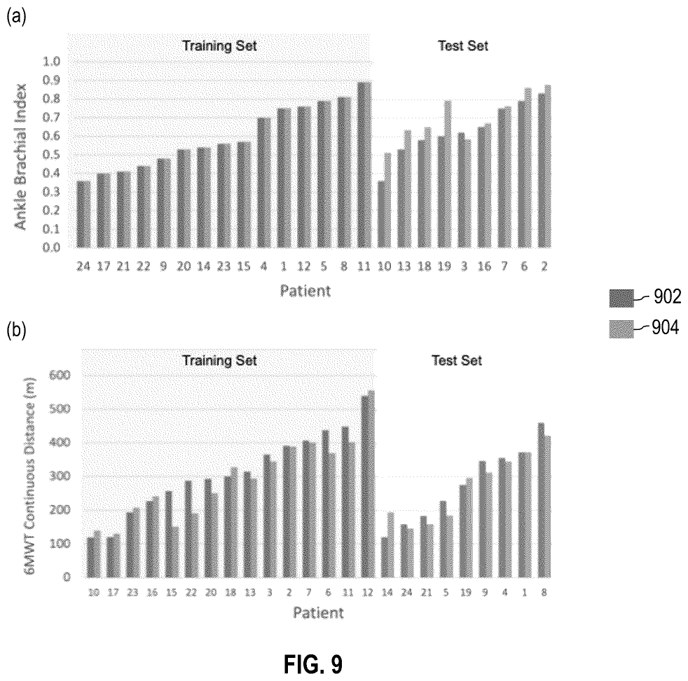

Results: FIG. 9 shows graphs indicating measured and predicted ankle-brachial index (ABI) and Six-Minute Walk Test (6MWT) Continuous Distance in individual patients in the training set and test set, according to some implementations. As shown in subpanels (a) and (b), measured values are shown as 902, and predicted values are shown as 904.

For patients in the test set, the ABI and 6MWT Continuous Distance predicted by the models differed from the actual measurements by 14%±13% and 15%±17%, respectively. For patients in the training set, the ABI and 6MWT Continuous Distance predicted by the models differed from the actual measurements by 010% and 12%±11%, respectively.

Discussion: From the above-discussed experiments, the VOT has the potential to predict the ABI and 6MWT Continuous Distance of patients diagnosed with PAD, suggesting that the VOT can be automated and used to monitor the severity of PAD. With more data from both healthy patients and PAD patients and improvement of the model, the VOT may complement ABI and 6MWT in the diagnosis and monitoring of PAD.

Peripheral arterial disease (PAD) is caused by a lack of blood flow to the musculature relative to its metabolism, which results in pain. PAD impacts up to 20% of patients around the world. PAD involves macrovascular and microvascular dysfunction. Near-infrared spectroscopy (NIRS) measures muscle oxygenation levels and assesses microvascular function. The standard of care for diagnosing PAD is the ankle-brachial index (ABI), which assesses macrovascular disease, and the 6-minute walk test (6MWT), which measures gait speed and claudication. NIRS has the potential to monitor the progression of PAD.

### Additional Experiment Set #3

A third study was to investigate whether NIRS measurement of muscle oxygenation during a standard test of vascular occlusion and post-occlusive hyperemia and the vascular occlusion test (VOT) is predictive of PAD severity as determined by ABI and 6MWT.

Using software that incorporates automated signal analysis and artificial neural networks, the third study assessed features of the NIRS StO2 signal derived from a vascular occlusion protocol related to skeletal muscle microvascular perfusion and metabolism in the context of PAD severity and functional capacity. The findings of the third study were three-fold. First, automated extraction of specific measures obtained from NIRS agreed with the same assessments performed manually, the current standard approach in NIRS research. Second, machine learning models developed in the present study were able to predict both ABI and % 6MWTd in PAD patients of varying severity with good-to-excellent agreement and accuracy. Finally, and in line with our initial hypothesis, these measures obtained from NIRS StO2 signals during a vascular occlusion protocol provided incremental information about PAD severity and functional performance beyond ABI.

The evaluation of PAD, both in terms of diagnosis and prognosis, is limited by the lack of rigorous clinical measures of microvascular function. This remains a critical area of concern across numerous diseases, including PAD where microvascular function is of paramount importance for several outcomes such as amputation. Even prior to serious adverse events, the management of PAD focuses heavily on cardiovascular risk factors (hypertension, obesity, etc.) that have well-established links to skeletal muscle and systemic microvascular health.

In the study, one objective was to extract features from a NIRS signal that included measurement of SmO₂ at baseline, during 5 minutes of occlusion of the calf muscles, and during post-ischemic recovery (often referred to as a Vascular Occlusion Test, or VOT) in patients with PAD. The study used an algorithm to extract key physiological features from the VOT signal and compared the features with conventional ABI and established measurements (continuous walking distance and total walking distance) obtained from the 6MWT. The study hypothesized that physiological features extracted from the NIRS signal would correlate with established clinical measurements of atherosclerosis in the leg arteries (ABI) and measurements of walking function (6MWT distances). NIRS assessment of SmO2 during the vascular occlusion test provides a reproducible assessment of oxygen delivery through the microcirculation as well as oxygen diffusivity and utilization by muscle. A finding of the study is that NIRS assessment of SmO₂ during the VOT provides an assessment of microvascular delivery function in the calf muscles of patients with PAD that correlates with measures of arterial perfusion (ABI) and walking function (6MWT distances) while also potentially providing an assessment of metabolic function in the calf muscles.

The health of skeletal muscle is an important determinant of disease progression, disability, and death, especially in older patients with declining mobility and in clinical populations that include patients with diabetes. In PAD, information yielded by NIRS provides an assessment of key pathophysiological determinants of disease progression, specifically oxygen delivery, and utilization in muscles of the lower limbs. NIRS profiles in PAD-affected lower extremities are characterized by rapid changes in oxygen saturation, significant hypoxia during exercise, and slow return to baseline oxygen saturation post-exercise. These profiles have a strong inverse correlation with walking capacity, independent of ABI. Numerous clinical studies have utilized NIRS to determine SmO₂ and to estimate muscle oxygen consumption during exercise or occlusion. Kragelj and colleagues demonstrated that the rate of reactive hyperemia and the maximal change during reactive hyperemia, both calculated from the muscle hemoglobin saturation signal of NIRS during an occlusive test, distinguished between healthy subject and patients with vascular disease. More than 25 years ago, McCully et al. reported that half-time to recovery of muscle oxygen saturation after exercise was significantly longer in patients with PAD as compared to age-matched healthy subjects. More recently, Gardner and colleagues compared calf muscle StO2, obtained during a standard treadmill test, with ambulatory function and health-related quality of life in patients with symptomatic PAD. These investigators found that a more rapid decline in muscle StO2 during a treadmill test was highly associated with worse ambulatory function and health-related quality of life. The studies demonstrate the value of NIRS for ongoing assessment of microvascular and metabolic function in the lower limbs of patients with PAD.

The data indicate a significant correlation between ABI and the rate of reperfusion of the calf muscles following 5 minutes of occlusion. The study also demonstrated a significant relationship between reperfusion rate and continuous and total walking distances in the 6MWT The findings support the notion that NIRS measurement of SmO₂ during the VOT is a valid assessment of microvascular perfusion and oxygen delivery that is compromised in patients with PAD. Agreement with the standard clinical measure of ABI as well as walking outcomes suggests that NIRS assessment during the VOT could be used predictively of functional status that is linked to microvascular dysfunction in addition to macrovascular disease.

Methods: Patients completed a 6-minute walk test (6MWT) to assess walking performance, according to the American Thoracic Society guidelines. Each patient was read, validated, and given standardized directions for the test. The patient was also instructed to notify when the onset of leg pain occurred during the 6MWT. Participants walked for 6 minutes back and forth over a distance of 30 meters marked by 2 cones, with a goal of covering as much distance as possible in the allotted time. If the participant needed to stop and rest during the test, the distance covered until the first stop was recorded as continuous walking distance. Total walking distance (TWD) was recorded in meters. Pain-free walk distance (PFWD) was recorded in meters as the distance before the onset of claudication pain. In addition, the percent distance achieved of the predicted 6-minute walk test distance based on age, sex, and body mass index, hereafter referred to as % 6MWTd, was determined.

The Walking Impairment Questionnaire (WIQ) was developed to assess perceived difficulty in walking distance, speed, and stair-climbing ability using multiple questions that were graded on a scale of 0-4, where 0 indicated inability and 4 indicated no difficulty. The distance score assessed difficulty walking different distances, from indoors to 1500 feet (5 blocks). The speed score measured difficulty walking one block at increasing speeds, from slow walking to jogging, while the stair-climbing score measured difficulty climbing one to three flights of stairs. The product of each domain was divided by the maximum possible score to obtain a percent score, wherein a higher score indicated a greater functional capacity.

NIRS imaging and vascular occlusion test setup: NIRS monitoring actively detected changes in tissue oxygen saturation (StO2) within small blood vessels, capillaries, and intracellular sites, assessing the balance of arterial inflow and oxygen utilization. StO₂ was continuously measured using a wireless continuous-wave NIRS spectrometer (Moxy monitoring systems, Hutchinson, Minnesota) with the probe placed in the middle of the belly of the lateral head of the gastrocnemius of the leg with the lowest ABI.

For each patient, StO₂ was measured during the ischemia-reperfusion paradigm, which included a 5-minute baseline, 5 minutes of proximal arterial occlusion, and 5 minutes after cuff deflation. Arterial occlusion was induced by inflating a pneumatic cuff (D. E. Hokanson, Bellevue, Wash) placed above the knee to 70-80 mmHg above the patient's systolic pressure, ensuring complete flow cessation to the lower limb. The absence of a tibial artery pulse confirmed successful occlusion.

NIRS signal analysis: Automated NIRS signal analysis was performed using Python v3.8.8. Briefly, StO2 signals were low-pass filtered (10^{th} order Butterworth filter, cutoff at 0.1 Hz) to remove noise. Using the filtered signal, specific features were automatically extracted. These features were baseline StO₂, the minimum StO₂ at end-ischemia, the maximum StO₂ achieved during reperfusion, the slope of StO₂ during occlusion, and the slope of StO₂ during the initial phase of reperfusion (i.e., the reperfusion slope). To quantify post-ischemic reperfusion, a reperfusion index (RI) was also determined, defined by the area under the StO₂ curve from cuff release until 2 minutes afterward, which incorporated both the rate and magnitude of post-ischemic reperfusion responses. These measures derived automatically from the exemplary system were compared to reference values calculated manually by an experienced technician blinded to patient characteristics. This blinding ensured unbiased assessments, allowing for accurate evaluation of any discrepancies between automated and technician-calculated values.

Machine learning and data analysis: To enhance the exemplary system (i.e., automated NIRS analysis software), deep neural networks were developed to analyze NIRS data, designed to streamline identifying and recognizing reperfusion abnormalities associated with PAD. Data from the exemplary system were collected for all patients and combined as input data. Output data were separately designated as ABI or the % 6MWTd. These data matrices were imported into Python v3.1 for the subsequent creation of the machine learning algorithm, which was constructed using PyTorch v2.5 and Keras v3.0.

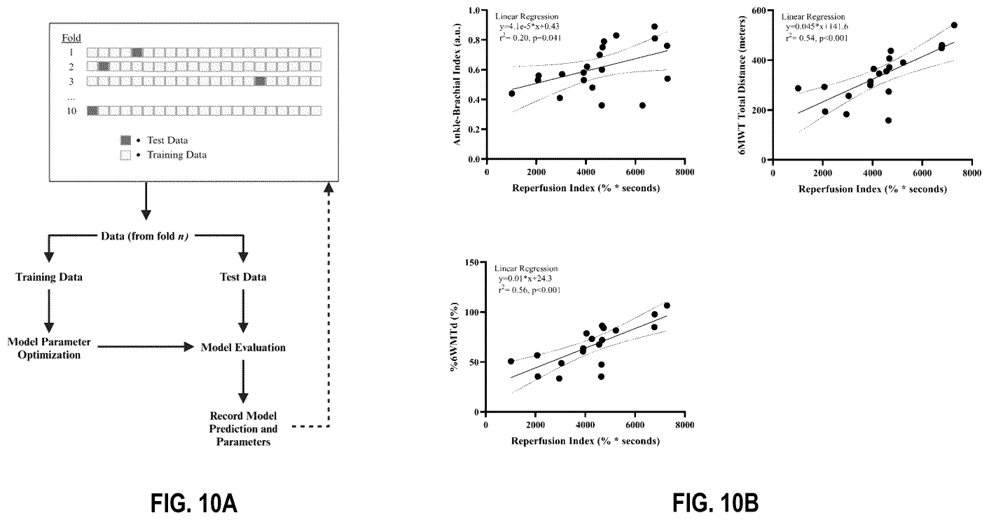

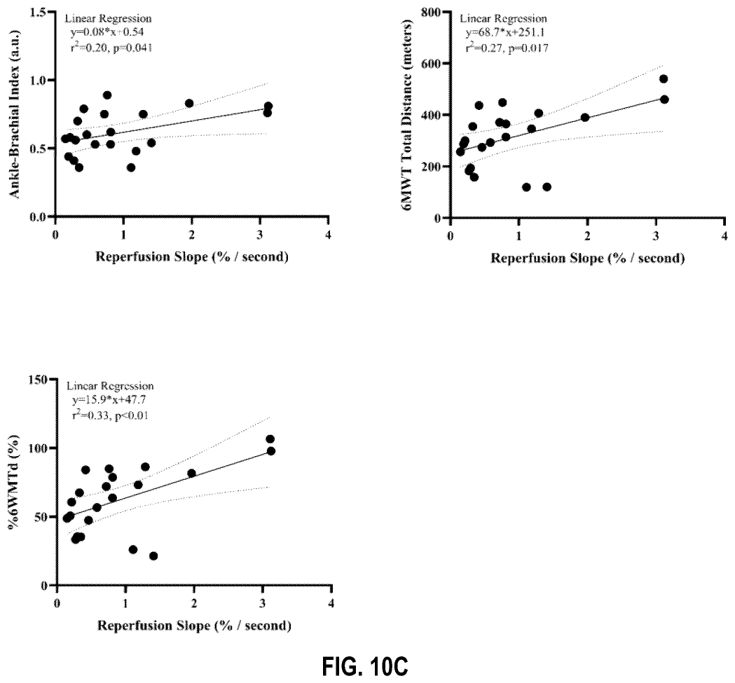

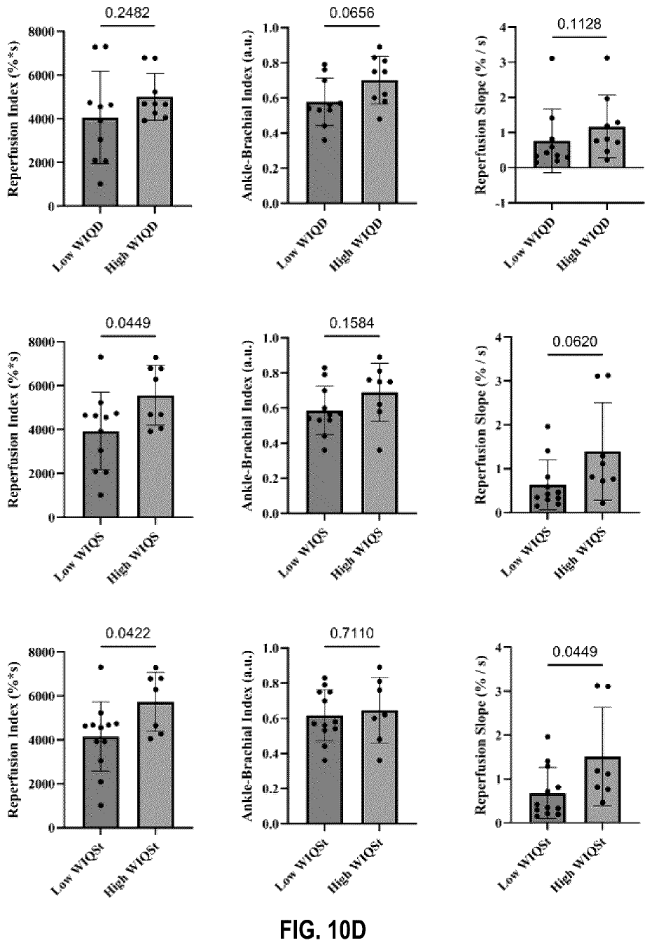

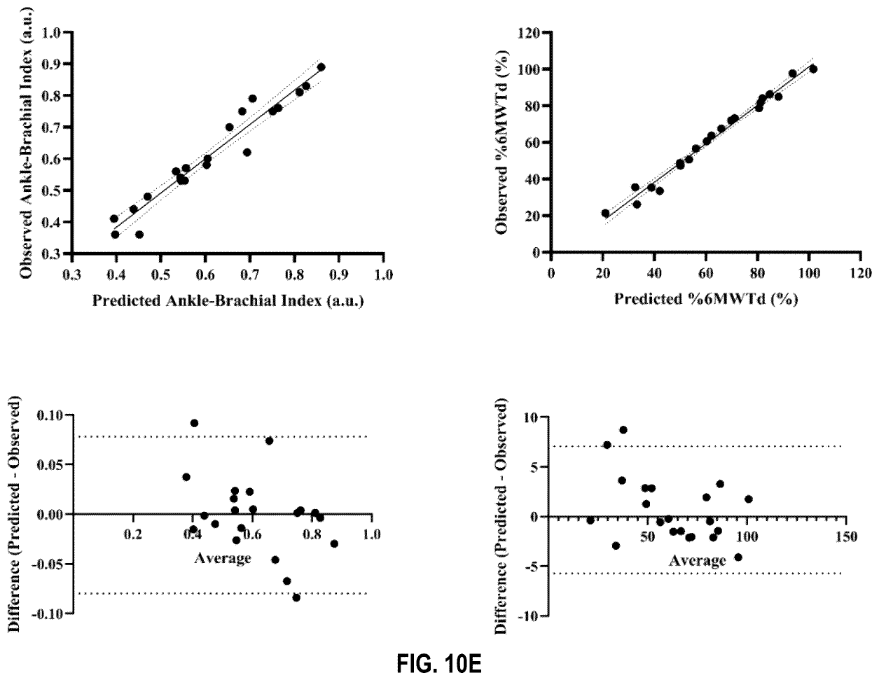

FIG. 10A shows a multi-fold leave-one-out cross-validation process. All tested algorithms were validated using multi-fold leave-one-out cross-validation, as has been described previously for physiologically relevant models, as shown in FIG. 10A. Each “fold” began with randomly allocating 1 patient to the test set and the remaining patients to the training set. Input and output data from the training set were used to train the model, with losses determined as the mean absolute error and an optimizer based on the Keras “RMSprop” algorithm. The trained model was used to predict the outcome for the 1 patient in the test set, and the prediction was recorded. Once this process was repeated so that each patient had been allocated to the test set, the results of each fold were compiled to determine the accuracy of the predictor neural network. The overall algorithm performance was assessed as a linear regression of the algorithm-predicted values versus the measured values. This allowed for the determination of accuracy (Pearson's correlation coefficient) and agreement of the magnitude of values (a linear slope near 1.0). The final machine learning model with the best performance for predicting ABI was a multilayer perceptron neural network consisting of 3 hidden layers, each layer containing 30 nodes that used the ‘relu6’ function and an output layer containing a single node with a ‘linear’ function. The final model with the best performance for predicting % 6MWTd was a multilayer perceptron neural network designed similarly to the ABI model.

In determining this final optimized model, additional tests were first performed to determine the impact of the number of nodes per hidden layer, followed by assessing the impact of different activation algorithms (i.e., ‘relu’ or ‘sigmoid’ functions versus the ‘relu6’ function). The final models described above and presented herein demonstrated the best performance across all models tested.

Demographics and group comparisons: All data were analyzed using commercially available software packages GraphPad v10.3.1 (San Diego, CA, USA) and STATA v18.5 (College Station, TX, USA). Independent-sample t-tests and Welch's t-test were used to determine pair differences between mild-to-moderate and severe PAD groups for normally distributed data with and without equal variance, respectively. Nonparametric Wilcoxon signed-rank tests were used to assess differences in non-normally distributed variables. Categorical data were compared using Fisher's exact test. Data were presented as mean and standard deviation (SD) or as 95% confidence intervals (CI) unless otherwise noted.

NIRS signal and outcome analyses: Agreement and consistency between manually calculated features of the NIRS StO2 signal and features automatically derived from the exemplary system were assessed using intraclass correlation coefficients (ICC). Linear regression was used to evaluate correlations between the RI and ABI, the distance covered during the 6-minute walk test, and the % 6MWTd. Outliers were assessed using the robust regression and outlier removal (ROUT) method, with Q set at 1%. To determine whether the NIRS reperfusion index (RI) provides incremental information beyond that offered by ABI, the RI and ABI were entered into a multivariate linear regression model with the same outcomes as the univariate regression models (6MWT total distance and % 6MWTd). For each outcome, the superior model (ABI alone vs ABI+RI) was determined using Akaike's information criterion. To assess the relationship between RI, ABI, and the WIQ scores in each domain, the cohort was split by each WIQ domain score median. RI and ABI were compared between the high and low WIQ score groups with unpaired t-tests (or Mann-Whitney nonparametric tests if applicable).

Machine learning models: In addition to calculating the slopes and Pearson correlation coefficients for the neural network-predicted ABI and % 6MWTd, Bland-Altman plots were used to determine the agreement between the predicted and measured outcomes. T-tests were also used to compare predicted and measured outcomes, and to assess the prediction bias.

Patient characteristics: Table 2 shows the patient demographics for 21 patients. All data are presented as mean±standard deviation for continuous data or number (percent) for categorical data.

TABLE 2
Patient demographics

| | Overall Cohort (n = 21) | |
|---|---|---|
| Age, years | 71.9 ± 8.3 | |
| Women, n (%) | 8 | (38.1) |
| Race/Ethnicity, n (%) | | |
| Non-Hispanic Black | 3 | (14.3) |
| Non-Hispanic White | 18 | (85.7) |
| BMI, kg/m² | 28.1 ± 5.2 | |
| SBP, mmHg | 135.3 ± 21.3 | |
| DBP, mmHg | 73.2 ± 10.9 | |
| Current Smokers, n (%) | 6 | (28.6) |
| Hypertension, n (%) | 16 | (76.2) |
| Diabetes Mellitus, n (%) | 6 | (28.6) |

| | |
|---|---|
| Ankle-brachial index, a.u. | 0.61 ± 0.16 |
| 6-minute walk test distance, meters | 315.1 ± 115.4 |
| Percent of predicted 6-minute walk test | 62.5 ± 24.0 |
| distance achieved, % | |
| WIQ Distance Score, % | 58.1 ± 32.2 |
| WIQ Speed Score, % | 53.6 ± 29.0 |
| WIQ Stair-Climbing Score, % | 60.1 ± 34.8 |

As shown in Table 2, there were 13 men and 8 women, with an age of 71.9±8.3 years (range 54-85 years). The mean ABI was 0.61±0.16 across all patients, 12 of whom (57%) had an ABI≤0.6. The mean total 6MWT distance was 315.1±115.4 meters, which was 62.5±24.0% of the predicted 6MWT distance. Functional impairment was prevalent in the cohort; the average WIQ distance score was 58.1±32.2%, the WIQ speed score was 53.6±29.0%, and the WIQ stair-climbing score was 60.1±34.8%.

When split by ABI≤0.6, there were no differences by PAD severity in age, sex distribution, body mass index (BMI), arterial blood pressure, smoking status, or presence of hypertension. Those who had severe PAD were less likely to have concomitant diabetes (p=0.02). The more severe group had a significantly lower 6MWT distance than the moderate group (237.0±22.7 vs 419.2±19.6 meters, p<0.01) and significantly lower % 6MWTd (46.0±4.6 vs 84.3±4.0%, p<0.01). This difference in functional capacity was supported by lower WIQ distance scores (42.2±9.2 vs. 75.8±8.9%, p=0.02), WIQ speed scores (38.2±8.6 vs. 70.9±6.9%, p=0.01), and WIQ stair-climbing scores (42.5±12.3 vs. 79.6±4.8%, p=0.02) in the severe PAD group.

NIRS responses and 6MWT outcomes: Table 3 shows the measurements from NIRS signals during vascular occlusion tests. All data were presented as mean±standard deviation. P-values were derived from t-tests (or Mann-Whitney tests, where appropriate); bolded p-values were significant at p<0.05.

TABLE 3
NIRS signals measurements during vascular occlusion tests.

| | Overall | Moderate | Severe | |
|---|---|---|---|---|
| | Cohort | PAD | PAD | p- |
| | (n = 21) | (n = 9) | (n = 12) | values |
| StO2 Baseline, % | 57.2 ± | 63.3 ± | 52.7 ± | 0.052 |
| | 12.6 | 10.8 | 12.2 | |
| StO2 Minimum, % | 11.0 ± | 11.9 ± | 10.4 ± | 0.77 |
| | 11.2 | 10.5 | 12.1 | |
| StO2 Maximum During | 70.9 ± | 75.0 ± | 67.9 ± | 0.064 |
| Reperfusion, % | 8.8 | 6.9 | 9.1 | |
| StO2 Occlusion | −0.17 ± | −0.18 ± | −0.16 ± | 0.096 |
| Slope, 0%/second | 0.06 | 0.03 | 0.07 | |
| StO2 Reperfusion | 0.93 ± | 1.39 ± | 0.59 ± | 0.041 |
| Slope, %/second | 0.87 | 1.09 | 0.44 | |
| Reperfusion Index | 4518.8 ± | 5417.9 ± | 3844.4 ± | 0.034 |
| (RI), % * seconds | 1720.7 | 1194.1 | 1785.8 | |

As shown in Table 3, automated analysis of the NIRS signals with the exemplary system demonstrated excellent consistency and agreement, with a range from the lowest ICC (0.72) for determining the reperfusion slope to the highest ICC (>0.99) for determining the maximal StO2 during the reperfusion phase. Therefore, all further results presented for NIRS analyses were those derived from the exemplary system.

FIG. 10B shows the relationship between the Reperfusion Index and the Ankle-Brachial Index, the Six-Minute Walk Test (6MWT) total distance, and the percentage of the predicted 6MWT distance (% 6MWTd). As shown in FIG. 10B, in univariate linear regression, the RI demonstrated significant relationships with ABI, 6MWT total distance, and % 6MWTd. When RI and ABI were both entered into a multivariate linear regression model for % 6MWTd, the model performed better than using ABI alone (difference in AIC: 0.70). The coefficient of determination (r²) for ABI alone was 0.65, whereas incorporating RI increased the model r² to 0.71. Conversely, the inclusion of RI into a model with ABI to predict 6MWT total distance did not improve model performance (difference in AIC: −0.58).

The above analyses were repeated for the reperfusion slope, a traditional measure used in NIRS vascular occlusion studies. FIG. 10C shows the relationship between reperfusion slope with Ankle-Brachial Index, Six-Minute Walk Test (6MWT) total distance, and percentage of the predicted 6MWT distance (% 6MWTd). As shown in FIG. 10C, the reperfusion slope was significantly associated with ABI, 6MWT total distance, and % 6MWTd. Including the reperfusion slope alongside ABI in multivariate regression models did not improve performance in the 6MWT total distance model (difference in AIC: −1.30). However, the reperfusion slope did add incremental value to ABI in the model for % 6MWTd (difference in AIC: 0.55).

NIRS responses and WIQ scores: FIG. 10D shows the WIQ domain scores for each domain (e.g., distance, speed, stair climbing) using predictor variables (RI, ABI, reperfusion slope). As shown in FIG. 10D, the cohort was stratified into high and low WIQ domain scores for each domain using the median score. None of the predictor variables differed between high and low WIQ distance scores (both p>0.05). RI differed between the high- and low-scoring groups for both the WIQ speed and WIQ stair-climbing scores, whereas the reperfusion slope only reached significance for the WIQ stair-climbing scores. ABI did not differ between groups in any domain.

Machine learning prediction of ABI: FIG. 10E shows the measured and predicted values of the Ankle-Brachial Index (ABI) and the percentage of the predicted 6MWT distance (% 6MWTd). As shown in FIG. 10E, the accuracy and agreement of the values predicted by the predictive model of the exemplary system were demonstrated by the correlation coefficient (r²=0.94) and a slope near 1.0 that included 1.0 in the 95% confidence interval (slope: 1.08, 95% CI: 0.95-1.21). Paired t-tests indicated no significant difference between measured and observed ABI (p=0.93). The Bland-Altman plot suggested minimal bias (−0.0008+SD 0.04) and good agreement (95% limits of agreement: −0.08-0.08).

Machine learning prediction of percent-achieved of predicted 6MWT distance: As shown in FIG. 10E, similarly to the ABI model, measurements of accuracy (r²=0.98) and agreement (slope: 1.05, 95% CI: 0.99-1.12) demonstrated strong predictive model performance of the exemplary system. Paired t-tests indicated no significant difference (p=0.36). Bland-Altman analyses indicated slight positive bias (0.66±SD 3.26) and good agreement (95% limits of agreement: −5.73-7.06).

Discussion Lower extremity peripheral artery disease (PAD) is a complex disease that affects over 200 million people worldwide, with a projected prevalence to double over the next decade. Unmitigated progression of this disease can manifest as severe functional limitations, exertional leg symptoms, limb-threatening ischemia, and subsequent amputation, which carries a mortality rate of >70% within 3 years. Unfortunately, such limb complications develop over time, even in asymptomatic patients, and are often poorly associated with conventional diagnostic tests, like the Ankle-Brachial Index and Doppler flow velocity (REFS) While such procedures provide accurate assessments of conduit artery occlusive pathology, they do not completely capture diffuse or small vessel disease, or microvascular dysfunction, which are now recognized as essential elements of PAD pathophysiology.

Over the past decade, a growing body of research has advanced our understanding of the microvascular basis for PAD-related outcomes. In patients with PAD with comorbid microvascular disease, there is an increased risk for lower-limb amputation and a higher risk of adverse and fatal events compared to PAD alone. In addition, clinical studies have applied several methods to define microvascular dysfunction related to PAD outcomes. Advanced procedures with magnetic resonance imaging (MRI), assessing tissue oxygenation and perfusion in response to the well-established ischemia-reperfusion paradigm, have revealed significantly impaired lower limb microvascular reactivity in individuals with PAD compared to matched controls with more significant microvascular dysfunction associated with PAD severity. Unfortunately, the use of MRI and related imaging techniques to diagnose microvascular dysfunction in the initial assessment of PAD microvascular is restricted in many settings due to its limited availability, high time commitment, and cost time.

Near-infrared spectroscopy (NIRS) is a clinically attractive alternative noninvasive optical imaging technique that quantifies tissue oxygenation reperfusion kinetics as an index of arterial inflow and oxygen utilization, and has shown potential in capturing the pathological changes within the microcirculation patients with PAD. Notably, NIRS permits evaluation of microvascular function in patients with PAD and could be of clinical interest in assessing PAD severity concurrently with ABI. The exemplary system using NIRS-derived ischemia-reperfusion response could detect PAD and evaluate its severity of symptoms and functional impairments independently of ABI. Given the need for accurate and timely quantification, conventional approaches to NIRS signal analysis in evaluating PAD rely on manual and subjective human analysis.

### Example Exercise Therapy

One of the more common symptoms, intermittent claudication, can impair walking function and physical activity, leading to further decline in cardiovascular health. Supervised exercise therapy has been linked to improvements in microvascular and walking function in the lower limb; however, its effectiveness in patients can be limited due to issues with accessibility and availability. There is a need for home-based therapy that improves microvascular function, oxygen delivery and related walking function in patients with peripheral artery disease.

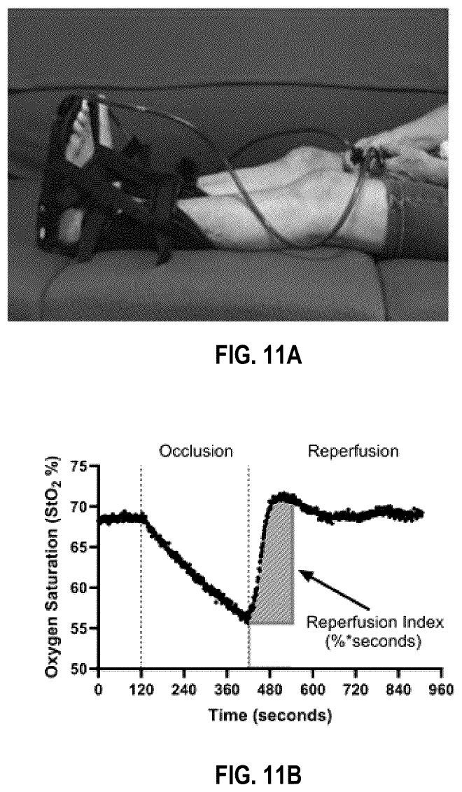

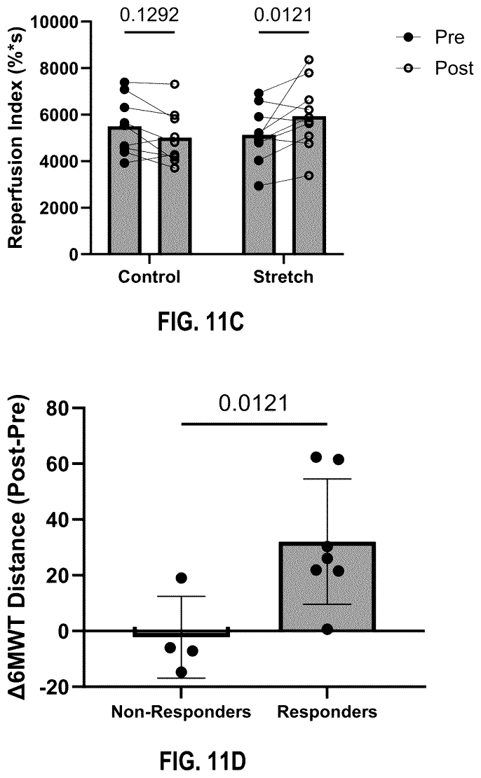

FIG. 11A shows example limb exercise. PAD patients were randomized to a stretch group or a placebo stretch group. Stretching was accomplished with a dorsiflexion splint, worn for 30 min/day, 5 days/week for 4 weeks. FIG. 11B shows results for a vascular occlusion test (VOT) and 6-minute walk test (6MWT) were administered before and after the intervention. Muscle StO2 was determined via Near-Infrared Spectroscopy (NIRS). FIG. 11C shows results of reperfusion Index (measured with NIRS during the VOT) increased in patients that performed 4 weeks of muscular stretching. FIG. 11D shows results for patients in which the amplitude of the muscle O2 signal improved after stretching also showed an increase in 6MWT distance.

The results shows that four weeks of daily muscle stretching can improve microvascular perfusion of muscle following occlusion, suggesting overall improved microvascular function. Improvement of microvascular perfusion, induced by daily muscular stretching, contributes to better matching of muscle O2 delivery to demand during walking, potentially leading to overall improvement in walking function in patients with peripheral artery disease. Results also shows the efficacy of the exemplary system and method in diagnosing and treating a microvascular dysfunction or disease of the extremity.

### Discussion

The first study incorporated the automated signal analysis and artificial neural networks into embodiments of the exemplary system to assess features of the NIRS StO2 signal derived from a vascular occlusion protocol related to skeletal muscle microvascular perfusion and metabolism in the context of PAD severity and functional capacity. The findings of the study were three-fold. First, the automated extraction of specific measures obtained from NIRS agreed with the same assessments performed manually, which is the current standard approach in NIRS research. Second, machine learning models developed in the study were able to predict both ABI and % 6MWTd in PAD patients of varying severity with good-to-excellent agreement and accuracy. Finally, these measures obtained from NIRS StO2 signals during a vascular occlusion protocol provided incremental information about PAD severity and functional performance beyond ABI

The evaluation of PAD, both in terms of diagnosis and prognosis, is limited by the lack of rigorous clinical measures of microvascular function. This remains an area of concern across numerous diseases, including PAD, where microvascular function is of paramount importance for several outcomes, such as amputation. Even prior to serious adverse events, the management of PAD focuses heavily on cardiovascular risk factors (hypertension, obesity, etc.) that have well-established links to skeletal muscle and systemic microvascular health.

Additional Discussion. Oxidative metabolism is intrinsically related to functional capacity. Impairment of oxidative metabolism in the muscles of the legs inevitably leads to reduced walking function, mobility, and quality of life. Reliable tools to investigate oxidative metabolism non-invasively in muscles of the lower limb of patients with PAD, with precision and reproducibility, may increase the ability to diagnose functional impairment as well as functional responsiveness to therapeutic and rehabilitative interventions. The experiments from the study suggested that there is a strong inverse relationship between both ABI and distances in the 6MWT and post-occlusive hyperemia. This inverse relationship may be indicative that as both microvascular and metabolic dysfunction progress in patients with PAD, there are limitations in the diffusivity and utilization of oxygen, resulting in a more prolonged overshoot of SmO₂ (above baseline) following release of occlusion. Further study is needed to compare the overshoot of SmO₂ with muscle energetics or with SmO₂ in the calves following discreet activation of the calf muscles, e.g., NIRS assessment of SmO₂ during calf raises.

NIRS has shown potential for early PAD detection, with patients experiencing a drop in oxygen saturation below baseline after an average of just 12 steps; however, standardization of NIRS results is hampered by low reproducibility of results obtained during walking. In addition, NIRS assessment could be a valuable means for evaluating the effectiveness of therapies used to treat PAD; however, NIRS remains underused in clinical practice. Although NIRS can provide insight into the pathophysiological state of patients with PAD, the variability in outcome parameters obtained with NIRS methodology hinders its implementation in clinical settings, either diagnostically or as a follow-up measurement during interventions. The use of NIRS for early detection of microvascular and muscle metabolic dysfunction could improve PAD diagnosis, enabling earlier intervention and potentially improving patient outcomes; however, a NIRS diagnostic that could be implemented in basic clinical practice with reliable and reproducible outcomes has not been developed. The study suggested that the utilization of a standardized protocol, the VOT, coupled with automated extraction of physiological features in the NIRS recording of SmO₂, could reduce variability in measurements and provide outcomes that could be universally applied to patients for diagnosis, prognosis, and follow-up during interventions.

### Configuration of Certain Implementations

The construction and arrangement of the systems and methods, as shown in the various implementations, are illustrative only. Although only a few implementations have been described in detail in this disclosure, many modifications are possible (e.g., variations in sizes, dimensions, structures, shapes, proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

Although the figures show a specific order of method steps, the order of the steps may differ from what is depicted. Also, two or more steps may be performed concurrently or with partial concurrence. Such variation will depend on the software and hardware systems chosen and on the designer's choice. All such variations are within the scope of the disclosure. Likewise, software implementations could be accomplished with standard programming techniques with rule-based logic and other logic to accomplish the various connection steps, processing steps, comparison steps, and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

The following patents, applications and publications as listed below and throughout this document are hereby incorporated by reference in their entirety herein.
[1] Tsao, C. W., et al., Heart Disease and Stroke Statistics—2023 Update: A Report From the American Heart Association. Circulation, 2023. 147 (8): p. e93-e621.
[1] Scully, R. E., et al., Estimated annual health care expenditures in individuals with peripheral arterial disease. Journal of Vascular Surgery, 2018. 67 (2): p. 558-567.
[2] Oliveira, D. C. d., et al., Association Between Ankle-Brachial Index and Coronary Lesions Assessed by Coronary Angiography: Cardiology Research, 2015. 6 (1): p. 216.
[3] Aboyans, V., et al., Measurement and Interpretation of the Ankle-Brachial Index. Circulation, 2012. 126 (24): p. 2890-2909.
[4] McDermott, M. M., et al., Functional Decline in Peripheral Arterial Disease Associations With the Ankle Brachial Index and Leg Symptoms. JAMA, 2004. 292 (4): p. 453-461.
[5] McDermott, M. M., et al., PROGNOSTIC VALUE OF FUNCTIONAL PERFORMANCE FOR MORTALITY IN PATIENTS WITH PERIPHERAL ARTERIAL DISEASE. Journal of the American College of Cardiology, 2008. 51 (15): p. 1482-1489.
[6] Papanas, N., et al., Ankle-brachial index: a surrogate marker of microvascular complications in type 2 diabetes mellitus? International Angiology: A Journal of the International Union of Angiology, 2007. 26 (3): p. 253-257.
[7] Baltrūnas, T., et al., The use of near-infrared spectroscopy in the diagnosis of peripheral artery disease: A systematic review. Vascular, 2022. 30 (4): p. 715-727.
[8] Barstow, T. J., Understanding near infrared spectroscopy and its application to skeletal muscle research. Journal of Applied Physiology, 2019. 126 (5): p. 1360-1376.
[9] Fuglestad, M. A., et al., A low-cost, wireless near-infrared spectroscopy device detects the presence of lower extremity atherosclerosis as measured by computed tomographic angiography and characterizes walking impairment in peripheral artery disease. Journal of Vascular Surgery, 2020. 71 (3): p. 946-957.
[10] Manfredini, F., et al., A dynamic objective evaluation of peripheral arterial disease by near-infrared spectroscopy. Eur J Vasc Endovasc Surg, 2009. 38 (4): p. 441-8.
[11] Ali, J., et al., Near-Infrared Spectroscopy (NIRS) for Cerebral and Tissue Oximetry: Analysis of Evolving Applications. J Cardiothorac Vasc Anesth, 2022. 36 (8 Pt A): p. 2758-2766.
[12] Barstow, T. J., Understanding near infrared spectroscopy and its application to skeletal muscle research. J Appl Physiol (1985), 2019. 126 (5): p. 1360-1376.
[13] Soares, R. N., et al., The association between near-infrared spectroscopy-derived and flow-mediated dilation assessment of vascular responsiveness in the arm. Microvasc Res, 2019. 122: p. 41-44.
[14] Cheatle, T. R., et al., Near-infrared spectroscopy in peripheral vascular disease. Br J Surg, 1991. 78 (4): p. 405-8.
[15] Van Beekvelt, M. C., et al., Performance of near-infrared spectroscopy in measuring local O(2) consumption and blood flow in skeletal muscle. J Appl Physiol (1985), 2001. 90 (2): p. 511-9.
[16] Laboratories, A.T.S.C.o.P.S.f.C.P.F., ATS statement: guidelines for the six-mimde walk test. Am J Respir Crit Care Med, 2002. 166 (1): p. 111-7.
[17] Jones, R.E.R.a.C.J., Measuring Functional Fitness of Older Adults. The Journal on Active Aging, 2002.
[18] Kragelj, R., T. Jarm, and D. Miklavcic, Reproducibility of parameters of postocclusive reactive hyperemia measured by near infrared spectroscopy and transcutaneous oximetry. Ann Biomed Eng, 2000. 28 (2): p. 168-73.
[19] McLeod, M., et al., Live strong and prosper: the importance of skeletal muscle strength for healthy ageing. Biogerontology, 2016. 17 (3): p. 497-510.
[20] Ruiz, J. R., et al., Association between muscular strength and mortality in men: prospective cohort study. BMJ, 2008. 337 (7661): p. a439.
[21] Vardi, M. and A. Nini, Near-infrared spectroscopy for evaluation of peripheral vascular disease. A systematic review of literature. Eur J Vasc Endovasc Surg, 2008. 35 (1): p. 68-74.
[22] Jarm, T., et al., Postocclusive reactive hyperemia in healthy volunteers and patients with peripheral vascular disease measured by three noninvasive methods. Adv Exp Med Biol, 2003. 530: p. 661-9.
[23] Kragelj, R., et al., Parameters of postocclusive reactive hyperemia measured by near infrared spectroscopy in patients with peripheral vascular disease and in healthy volunteers. Ann Biomed Eng, 2001. 29(4): p. 311-20.
[24] McCully, K. K., C. Halber, and J. D. Posner, Exercise-induced changes in oxygen saturation in the calf muscles of elderly subjects with peripheral vascular disease. J Gerontol, 1994. 49 (3): p. B128-34.
[25] Gardner, A. W., et al., Association between calf muscle oxygen saturation with ambulatory function and quality of life in symptomatic patients with peripheral artery disease. J Vasc Surg, 2020. 72 (2): p. 632-642.
[26] Cornelis, N., et al., The Use of Near Infrared Spectroscopy to Evaluate the Effect of Exercise on Peripheral Muscle Oxygenation in Patients with Lower Extremity Artery Disease: A Systematic Review. Eur J Vasc Endovasc Surg, 2021. 61 (5): p. 837-847.
[27] McLay, K. M., et al., Repeatability of vascular responsiveness measures derived from near-infrared spectroscopy. Physiol Rep, 2016. 4 (9).
[28] Ali J, Cody J, Maldonado Y, Ramakrishna H. Near-Infrared Spectroscopy (NIRS) for Cerebral and Tissue Oximetry: Analysis of Evolving Applications. J Cardiothorac Vasc Anesth 36:2758-2766, 2022.
[29] Barstow T J. Understanding near infrared spectroscopy and its application to skeletal muscle research. J Appl Physiol (1985) 126:1360-1376, 2019.
[30] McLay K M, Nederveen J P, Pogliaghi S, Paterson D H, Murias J M. Repeatability of vascular responsiveness measures derived from near-infrared spectroscopy. Physiol Rep 4:2016.
[31] Soares R N, Somani Y B, Proctor D N, Murias J M. The association between near-infrared spectroscopy-derived and flow-mediated dilation assessment of vascular responsiveness in the arm. Microvasc Res 122:41-44, 2019.
[32] Jones W S, Patel M R, Dai D, Vemulapalli S, Subherwal S, Stafford J, Peterson E D. High mortality risks after major lower extremity amputation in Medicare patients with peripheral artery disease. Am Heart J. 2013 May; 165 (5): 809-15, 815.el. doi: 10.1016/j.ahj.2012.12.002. Epub 2013 Feb. 5. PMID: 23622919.
[33] Faxon D P, Fuster V, Libby P, Beckman J A, Hiatt W R, Thompson R W, Topper J N, Annex B H, Rundback J H, Fabunmi R P, Robertson R M, Loscalzo J; American Heart Association. Atherosclerotic Vascular Disease Conference: Writing Group III: pathophysiology. Circulation. 2004 Jun. 1;109 (21): 2617-25. doi: 10.1161/01.CIR.0000128520.37674.EF. PMID: 15173044.
[34] Faxon D P, Creager M A, Smith S C Jr, Pasternak R C, Olin J W, Bettmann M A, Criqui M H, Milani R V, Loscalzo J, Kaufman J A, Jones D W, Pearce W H; American Heart Association. Atherosclerotic Vascular Disease Conference: Executive summary: Atherosclerotic Vascular Disease Conference proceeding for healthcare professionals from a special writing group of the American Heart Association. Circulation. 2004 Jun. 1: 109 (21): 2595-604. doi: 10.1161/01.CIR.0000128517.52533.DB. PMID: 15173041.
[35] Hiatt W R, Armstrong E J, Larson C J, Brass E P. Pathogenesis of the limb manifestations and exercise limitations in peripheral artery disease. Circ Res. 2015 Apr. 24; 116 (9): 1527-39. doi: 10.1161/CIRCRESAHA.116.303566. PMID: 25908726.
[36] Beckman J A, Duncan M S, Damrauer S M, Wells Q S, Barnett J V, Wasserman D H, Bedimo R J, Butt A A, Marconi V C, Sico J J, Tindle H A, Bonaca M P, Aday A W, Freiberg M S. Microvascular Disease, Peripheral Artery Disease, and Amputation. Circulation. 2019 Aug. 6;140 (6): 449-458. doi: 10.1161/CIRCULATIONAHA.119.040672. Epub 2019 Jul. 8. PMID: 31280589; PMCID: PMC6682431.
[37] Bunte M C, Jacob J, Nudelman B, Shishehbor M H. Validation of the relationship between ankle-brachial and toe-brachial indices and infragenicular arterial patency in critical limb ischemia. Vasc Med. 2015 February;20 (1): 23-9. doi: 10.1177/1358863X14565372. Epub 2015 Jan. 28. PMID: 25630991.
[38] Sukul D, Grey S F, Henke P K, Gurm H S, Grossman P M. Heterogeneity of Ankle-Brachial Indices in Patients Undergoing Revascularization for Critical Limb Ischemia. JACC Cardiovasc Interv. 2017 Nov. 27;10 (22): 2307-2316. doi: 10.1016/j.jcin.2017.08.026. PMID: 29169498; PMCID: PMC6800014.
[39] Sukul D, Grey S F, Henke P K, Gurm H S, Grossman P M. Heterogeneity of Ankle-Brachial Indices in Patients Undergoing Revascularization for Critical Limb Ischemia. JACC Cardiovasc Interv. 2017 Nov. 27;10 (22): 2307-2316. doi: 10.1016/j.jcin.2017.08.026. PMID: 29169498; PMCID: PMC6800014.
[40] Yang C, Kwak L, Ballew S H, Jaar B G, Deal J A, Folsom A R, Heiss G, Sharrett A R, Selvin E, Sabanayagam C, Coresh J, Matsushita K. Retinal microvascular findings and risk of incident peripheral artery disease: An analysis from the Atherosclerosis Risk in Communities (ARIC) Study. Atherosclerosis. 2020 February;294:62-71. doi: 10.1016/j.atherosclerosis.2019.10.012. Epub 2019 Oct. 11. PMID: 31812251; PMCID: PMC7012766.
[41] Grubman S, Algara M, Smolderen K G, Luna P, Walenczyk K, Scierka L, Cleman J, Siddiqui W T, Romain G, Mena-Hurtado C. Examining Outcomes in Patients Admitted With Comorbid Peripheral Artery Disease and Microvascular Disease. J Am Heart Assoc. 2024 Jan. 2: 13 (1): e030710. doi: 10.1161/JAHA.123.030710. Epub 2024 Jan. 3. PMID: 38166496; PMCID: PMC10863818.
[42] Baltrünas T, Mosenko V, Mackevičius A, Dambrauskas V, Ašakienė I, Ručinskas K, Narmontas P. The use of near-infrared spectroscopy in the diagnosis of peripheral artery disease: A systematic review. Vascular. 2022 August: 30 (4): 715-727. doi: 10.1177/17085381211025174. Epub 2021 Jun. 10. PMID: 34112030.
[43] Barstow T J. Understanding near infrared spectroscopy and its application to skeletal muscle research. J Appl Physiol (1985). 2019 May 1;126 (5): 1360-1376. doi: 10.1152/japplphysiol.00166.2018. Epub 2019 Mar. 7. PMID: 30844336.
[44] Englund E K, Langham M C, Ratcliffe S J, Fanning M J, Wehrli F W, Mohler E R 3rd, Floyd T F. Multiparametric assessment of vascular function in peripheral artery disease: dynamic measurement of skeletal muscle perfusion, blood-oxygen-level dependent signal, and venous oxygen saturation. Circ Cardiovasc Imaging. 2015 April;8 (4): 10.1161/CIRCIMAGING.114.002673 e002673. doi: 10.1161/CIRCIMAGING. 114.002673. PMID: 25873722; PMCID: PMC4399002.
[45] Wu W C, Mohler E 3rd, Ratcliffe S J, Wehrli F W, Detre J A, Floyd T F. Skeletal muscle microvascular flow in progressive peripheral artery disease: assessment with continuous arterial spin-labeling perfusion magnetic resonance imaging. J Am Coll Cardiol. 2009 Jun. 23;53 (25): 2372-7. doi: 10.1016/j.jacc.2009.03.033. PMID: 19539149; PMCID: PMC2763280.
[46] Fuglestad M A, Hernandez H, Gao Y, Ybay H, Schieber M N, Brunette K E, Myers S A, Casale G P, Pipinos I I. A low-cost, wireless near-infrared spectroscopy device detects the presence of lower extremity atherosclerosis as measured by computed tomographic angiography and characterizes walking impairment in peripheral artery disease. J Vasc Surg. 2020 March; 71 (3): 946-957. doi: 10.1016/j.jvs.2019.04.493. Epub 2019 Aug. 21. PMID: 31445826; PMCID: PMC7501596.
[47] Joseph S, Munshi B, Agarini R, Kwok R C H, Green D J, Jansen S. Near infrared spectroscopy in peripheral artery disease and the diabetic foot: A systematic review. Diabetes Metab Res Rev. 2022 October: 38 (7): e3571. doi: 10.1002/dmrr.3571. Epub 2022 Aug. 17. PMID: 35939767.
[48] Vardi M, Nini A. Near-infrared spectroscopy for evaluation of peripheral vascular disease. A systematic review of literature. Eur J Vasc Endovasc Surg. 2008 January;35 (1): 68-74. doi: 10.1016/j.ejvs.2007.07.015. Epub 2007 Oct. 4. PMID: 17919945.
[49] ATS Committee on Proficiency Standards for Clinical Pulmonary Function Laboratories. ATS statement: guidelines for the six-minute walk test. Am J Respir Crit Care Med. 2002 Jul. 1;166 (1): 111-7. doi: 10.1164/ajrccm. 166.1 at1102. Erratum in: Am J Respir Crit Care Med. 2016 May 15;193 (10): 1185. doi: 10.1164/rccm.19310erratum. PMID: 12091180.
[50] Britto R R, Probst V S, de Andrade A F, Samora G A, Hernandes N A, Marinho P E, Karsten M, Pitta F, Parreira V F. Reference equations for the six-minute walk distance based on a Brazilian multicenter study. Braz J Phys Ther. 2013 November-December; 17 (6): 556-63. doi: 10.1590/S1413-35552012005000122. Epub 2013 Nov. 14. PMID: 24271092; PMCID: PMC4207140.
[51] Ritti-Dias R M, Sant'anna FDS, Braghieri H A, Wolosker N, Puech-Leao P, Lanza F C, Cucato G G, Dal Corso S, Correia M A. Expanding the Use of Six-Minute Walking Test in Patients with Intermittent Claudication. Ann Vasc Surg. 2021 January; 70:258-262. doi: 10.1016/j.avsg.2020.07.047. Epub 2020 Aug. 13. PMID: 32800882.
[52] Regensteiner J G, Steiner J F, Hiatt W R. Exercise training improves functional status in patients with peripheral arterial disease. J Vasc Surg. 1996 January;23 (1): 104-15. doi: 10.1016/s0741-5214 (05) 80040-0. PMID: 8558725.
[53] Jain A, Liu K, Ferrucci L, Criqui M H, Tian L, Guralnik J M, Tao H, McDermott M M. The Walking Impairment Questionnaire stair-climbing score predicts mortality in men and women with peripheral arterial disease. J Vasc Surg. 2012 June;55 (6): 1662-73.e2. doi: 10.1016/j.jvs.2011.12.010. PMID: 22608041; PMCID: PMC3963605.
[54] Jain A, Liu K, Ferrucci L, Criqui M H, Tian L, Guralnik J M, Tao H, McDermott M M. Declining walking impairment questionnaire scores are associated with subsequent increased mortality in peripheral artery disease. J Am Coll Cardiol. 2013 Apr. 30;61 (17): 1820-9. doi: 10.1016/j.jacc.2013.01.060. Epub 2013 Feb. 28. PMID: 23500321; PMCID: PMC6116834.
[55] Grieger D M, Brandt K E, Diekman M A. Follicular fluid concentrations of estradiol-17 beta and progesterone and secretory patterns of LH and FSH in prepubertal gilts reared in confinement or outdoor lots. J Anim Sci. 1986 March;62 (3): 751-8. doi: 10.2527/jas1986.623751x. PMID: 3084436.
[56] Feldmann A, Schmitz R, Erlacher D. Near-infrared spectroscopy-derived muscle oxygen saturation on a 0% to 100% scale: reliability and validity of the Moxy Monitor. J Biomed Opt. 2019 November;24 (11): 1-11. doi: 10.1117/1.JBO.24.11.115001. PMID: 31741352; PMCID: PMC7003144.
[57] McManus C J, Collison J, Cooper C E. Performance comparison of the MOXY and PortaMon near-infrared spectroscopy muscle oximeters at rest and during exercise. J Biomed Opt. 2018 January;23 (1): 1-14. doi: 10.1117/1.JBO.23.1.015007. PMID: 29368457.
[58] Rodriguez R F, Townsend N E, Aughey R J, Billaut F. Influence of averaging method on muscle deoxygenation interpretation during repeated-sprint exercise. Scand J Med Sci Sports. 2018 November;28 (11): 2263-2271. doi: 10.1111/sms.13238. Epub 2018 Jun. 28. PMID: 29883534.
[59] Ramos Gonzalez M, Caldwell J T, Branch P A, Wardlow G C, Black C D, Campbell J, Larson R D, Ade C J. Impact of shear rate pattern on post-occlusive near-infrared spectroscopy microvascular reactivity. Microvasc Res. 2018 March; 116:50-56. doi: 10.1016/j.mvr.2017.10.004. Epub 2017 Nov. 6. PMID: 29080792.
[60] Xu D, Zhang S, Zhang H, Mandic D P. Convergence of the RMSProp deep learning method with penalty for nonconvex optimization. Neural Netw. 2021 July; 139:17-23. doi: 10.1016/j.neunet.2021.02.011. Epub 2021 Feb. 23. PMID: 33662649.
[61] Beltrame T, Amelard R, Villar R, Shafiee M J, Wong A, Hughson R L. Estimating oxygen uptake and energy expenditure during treadmill walking by neural network analysis of easy-to-obtain inputs. J Appl Physiol (1985). 2016 Nov. 1;121 (5): 1226-1233. doi: 10.1152/japplphysiol.00600.2016. Epub 2016 Sep. 29. PMID: 27687561.

What is claimed is:

1. A system comprising:
at least one processor; and
memory having instructions stored thereon that, when executed by the at least one processor, cause the system to:
obtain a re-perfused oxygen saturation signal (e.g., near-infrared spectroscopy signal) recorded from a second position at an extremity of the subject (e.g., during a vascular occlusion test (VOT)) downstream to a first position having positioned a pressure cuff to cause occlusion of the extremity when inflated;
generate, via a trained AI model, or a model derived therefrom, a perfusion index value or score, wherein the trained AI model was trained based on a microvascular function assessment and re-perfused oxygen saturation signal; and
output the perfusion index value or score or a parameter derived therefrom as an output of the trained AI model, wherein the output is presented in a graphical user interface or report for use by a clinician to diagnose or treat a microvascular dysfunction or disease of the extremity.

2. The system of claim 1, wherein the instructions further cause the system to:
cause an auditory or visual alert to be generated at the graphical user interface if a microvascular dysfunction is detected.

3. The system of claim 2, wherein the alert comprises an indication of a type of microvascular dysfunction, wherein the type of microvascular dysfunction is predicted from the perfusion index.

4. The system of claim 1, wherein the instructions further cause the system to:
preprocess the re-perfused oxygen saturation signal to remove outliers (e.g., using a seven-tap outlier detection filter) prior to using the trained AI model to generate the perfusion index value or score.

5. The system of claim 1, further comprising:
a pressure cuff configured to be positioned at the first position; and
direct control output for inflation and deflation of the pressure cuff in accordance with a predefined test procedure (e.g., in a VOT test).

6. The system of claim 5, further comprising:
a near-infrared spectroscopy device configured to acquire the re-perfused oxygen saturation signal.

7. The system of claim 1, wherein the trained AI model employs one or more ML features selected from the group consisting of:
a first feature associated with a rate of oxygen utilization during a period of occlusion;
a second feature associated with a rate of microvascular reperfusion upon release of occlusion;
a third feature associated with a hyperemic response of muscle microvasculature;
a fourth feature associated with a rate of return of oxygen saturation to baseline after occlusion; and
a fifth feature associated with overall oxygenation of muscle during the occlusion of the extremity.

8. The system of claim 1, wherein the trained AI model is a neural network.

9. A method for estimating microvascular health in an extremity of a subject, the method comprising:
obtaining by a processor a re-perfused oxygen saturation signal (e.g., near-infrared spectroscopy signal) recorded from a second position at the extremity of the subject (e.g., during a vascular occlusion test (VOT)) downstream to a first position having positioned a pressure cuff to cause occlusion of the extremity when inflated;
generating, via a trained AI model, or a model derived therefrom, a perfusion index value or score, wherein the trained AI model was trained based on a microvascular function assessment and re-perfused oxygen saturation signal; and
outputting the perfusion index value or score or a parameter derived therefrom as an output of the trained AI model, wherein the output is presented in a graphical user interface or report for use by a clinician to diagnose or treat a microvascular dysfunction or disease of the extremity.

10. The method of claim 9, further comprising:
determining whether the estimated microvascular function is indicative of microvascular dysfunction, wherein the indication comprises an alert if microvascular dysfunction is detected.

11. The method of claim 10, wherein the alert comprises an indication of a type of microvascular dysfunction, wherein the type of microvascular dysfunction is predicted from the perfusion index.

12. The method of claim 9, further comprising:
preprocessing the re-perfused oxygen saturation signal to remove outliers (e.g., using a seven-tap outlier detection filter) prior to using the trained AI model to generate the perfusion index value or score.

13. The method of claim 9, further comprising:
controlling inflation and deflation of the pressure cuff in accordance with a predefined test procedure.

14. The method of claim 9 wherein a near-infrared spectroscopy device acquired the re-perfused oxygen saturation signal.

15. The method of claim 9, wherein the trained AI model employs one or more ML features selected from the group consisting of:
a first feature associated with a rate of oxygen utilization during a period of occlusion;
a second feature associated with a rate of microvascular reperfusion upon release of occlusion;
a third feature associated with a hyperemic response of muscle microvasculature;
a fourth feature associated with a rate of return of oxygen saturation to baseline after occlusion; and
a fifth feature associated with overall oxygenation of muscle during the occlusion of the extremity.

16. A non-transitory computer readable medium having instructions for estimating microvascular health in an extremity of a subject, the instructions when executed by a processor causes the processor to:
obtain a re-perfused oxygen saturation signal (e.g., near-infrared spectroscopy signal) recorded from a second position at the extremity of the subject (e.g., during a vascular occlusion test (VOT)) downstream to a first position having positioned a pressure cuff to cause occlusion of the extremity when inflated;
generate, via a trained AI model, or a model derived therefrom, a perfusion index value or score, wherein the trained AI model was trained based on a microvascular function assessment and re-perfused oxygen saturation signal; and
output the perfusion index value or score or a parameter derived therefrom as an output of the trained AI model, wherein the output is presented in a graphical user interface or report for use by a clinician to diagnose or treat a microvascular dysfunction or disease of the extremity.

17. The non-transitory computer readable medium of claim 16, wherein the instructions when executed by the processor further causes the processor to determine whether the estimated microvascular function is indicative of microvascular dysfunction, wherein the indication comprises an alert if microvascular dysfunction is detected.

18. The non-transitory computer readable medium of claim 17, wherein the alert comprises an indication of a type of microvascular dysfunction, wherein the type of microvascular dysfunction is predicted from the perfusion index.

19. The non-transitory computer readable medium of claim 16, wherein the instructions when executed by the processor further causes the processor to control inflation and deflation of the pressure cuff in accordance with a predefined test procedure.

20. The non-transitory computer readable medium of claim 16, wherein a near-infrared spectroscopy device acquired the re-perfused oxygen saturation signal.
